(19) United States
(12) Patent Application Publication
(10) Pub. No.: US 2024/0025994 A1
(43) Pub. Date: Jan. 25, 2024

(54) ANTI-LAG-3 MONOCLONAL ANTIBODY AND ANTIGEN BINDING FRAGMENT THEREOF, AND USE THEREOF

(71) Applicants: BEIJING DONGFANG BIOTECH CO., LTD, Beijing (CN); BEIJING JINGYITAIXIANG TECHNOLOGY DEVELOPMENT CO. LTD., Beijing (CN)

(72) Inventors: YI BAI, Beijing (CN); Junjie ZHOU, Beijing (CN); Si LIU, Beijing (CN)

(21) Appl. No.: 18/039,720

(22) PCT Filed: Nov. 29, 2021
(86) PCT No.: PCT/CN2021/133941

§ 371 Date: Jun. 1, 2023

(30) Foreign Application Priority Data
Dec. 10, 2020 (CN) 202011436581.5

(51) Int. Cl. C07K 16/28; C12N 15/63; A61P 35/00

(52) U.S. Cl. CPC C07K 16/2803; C12N 15/63; C07K 16/2818; A61P 35/00; C07K 2317/565; C07K 2317/24

(57) ABSTRACT

The present application relates to the field of biomedicine, and particularly provides an anti-LAG-3 monoclonal antibody and an antigen binding fragment thereof, and use thereof, wherein the anti-LAG-3 monoclonal antibody and the antigen binding fragment thereof comprise a heavy chain variable region and a light chain variable region, and are selected from any one of A-1, A-2: A-3, and A-4.

### CROSS-REFERENCE TO RELATED APPLICATION

The present application is a National Stage of International Patent Application No. PCT/CN20211133941, filed on Nov. 29, 2021, and claims priority of Chinese patent application 202011436581.5 filed on Dec. 10, 2020, which is incorporated by reference herein.

### SEQUENCE LISTING

The instant disclosure contains a Sequence Listing which has submitted electronically in ASCII format and is hereby incorporated by reference in its entirety. Said ASCII copy is named PN210936 SEQ LIST.txt and is 45258 bytes in size. The sequence listing contains 47 sequences, which is identical in substance to the sequences disclosed in the PCT application, except the comments for the artificial sequences have been added, and includes no new matter.

### Technical Field

The present disclosure relates to the technical filed of biological medicine, and particularly relates to an anti-LAG-3 monoclonal antibody and an antigen binding fragment thereof, and use thereof.

### Background

Immunotherapy is a most popular cancer treatment mode at present and known as a third revolution of cancer treatment. The so-called “cancer immunotherapy” is a therapeutic method for attacking cancer cells by means of an autoimmune system of human body. A balance between the immune system and the cancer cells is a dynamic process of long-term gambling, aid is both positively engaged and interwoven. Immune cells of a healthy body may discover and kill the cancer cells, but under an induction of various innate and acquired factors, the immune system may lose an absolute advantage and is even “instigated” by the cancer cells, and helps the occurrence and development of cancer. A targeting drug has been hoped for its accuracy, but cancer cells are prone to develop drug resistance due to their fickle and complexity. Therefore, the accuracy is also easily useless. For this reason, immune checkpoint inhibitors in the cancer treatment have gradually become a research hotspot since CTLA-4 and PD-1 are discovered.

With the advent of an immunological anti-cancer therapy, lymphocyte-activation gene 3 (LAG-3) is gradually recognized as a potential immune checkpoint receptor. It is reported that the LAG-3 plays an important role in promoting an activity of regulatory T cells and down-regulating activation and proliferation of T cells (Workman CJ et al., J. Immunol., 2005, 174: 688-695). The LAG-3 is also known as CD223. The gene comprises 8 exam, and is located on human chromosome 12 (mouse chromosome 6). The LAG-3 belongs to the immunoglobulin superfamily, and consists of 3 parts of an extracellular region, a transmembrane region, and a cytoplasmic region. The gene encodes a type I transmembrane protein consisting of 498 amino acids. The LAG-3 is widely expressed on surfaces of activated NK cells, T cells and other immune cells, may negatively regulate lymphocyte functions, and has been proved by a part of studies to play an important role in the aspects of tumors and autoimmune diseases.

Preclinical studies show that inhibition of the LAG-3 enables T cells to regain cytotoxicity, thereby limiting growth of tumors and enhancing a killing effect of tumors. At the same time, the inhibition of the LAG-3 also reduces an ability of regulatory T cells in inhibiting an immune response. Therefore, the LAG-3 is an immune checkpoint receptor that may regulate functions of T cells, the inhibition of the LAG-3 is more beneficial to patients, particularly those with tumors containing LAG-3-expressing immune cells. Thus, the LAG-3 is considered a more attractive target than other immune checkpoint proteins. In the current second-generation immune checkpoint targets, the LAG-3 is a target with more clinical data and relatively determined druggability. In view of the above importance of the existence of the LAG-3, in order to meet needs of patients with tumors or other immunological diseases at home and abroad, it is urgently needed to develop an anti-LAG-3 monoclonal antibody with a relatively high biological activity.

### SUMMARY

In order to meet needs of the domestic market, the present disclosure obtains an anti-LAG-3 monoclonal antibody or an antigen binding fragment thereof which may specifically bind to LAG-3 and has a relatively high biological activity through screening of an immune library.

Specific technical solutions of the present disclosure are as follows:

The present disclosure provides an anti-LAG-3 monoclonal antibody or an antigen binding fragment thereof, comprising a heavy chain variable region and a light chain variable region, wherein the heavy chain variable region comprises 3 heavy chain complementarily determining regions, respectively represented by HCDR1, HCDR2, and HCDR3, and the light chain variable region comprises 3 light chain complementarily determining regions, respectively represented by LCDR1, LCDR2, and LCDR3, and the monoclonal antibody or the antigen binding fragment thereof is selected from any one of the following.

A-1: the heavy chain complementarily determining region HCDR1 comprises an amino acid sequence shown in SEQ ID No: 1, the heavy chain complementarily determining region HCDR2 comprises an amino acid sequence shown in SEQ ID No: 2, the heavy chain complementarily determining region HCDR3 comprises an amino acid sequence shown in SEQ ID No: 3, the light chain complementarily determining region LCDR1 comprises an amino acid sequence shown in SEQ ID No: 4, the light chain complementarily determining region LCDR2 comprises an amino acid sequence shown in SEQ ID No: 5, and the light chain complementarily determining region LCDR3 comprises an amino acid sequence shown in SEQ ID No: 6;

A-2. the heavy chain complementarity determining region HCDR1 comprises an amino acid sequence shown in SEQ ID No: 1, the heavy chain complementarity determining region HCDR2 comprises an amino acid sequence shown in SEQ ID No: 2, the heavy chain complementarily determining region HCDR3 comprises an amino acid sequence shown in SEQ ID No: 3, the light chain complementarily determining region LCDR1 comprises an amino acid sequence shown in SEQ ID No: 4, the light chain complementarity determining region LCDR2 comprises an amino acid sequence shown in SEQ ID No: 7, and the light chain complementarity determining region LCDR3 comprises an amino acid sequence shown in SEQ ID No: 8;

A-3. the heavy chain complementarily determining region HCDR1 comprises an amino acid sequence shown in SEQ ID No: 9, the heavy chain complementarity determining region HCDR2 comprises an amino acid sequence shown in SEQ ID No: 10, the heavy chain complementarily determining region HCDR3 comprises an amino acid sequence shown in SEQ ID No: 11, the light chain complementarity determining region LCDR1 comprises an amino acid sequence shown in SEQ ID No: 12, the light chain complementarily determining region LCDR2 comprises an amino acid sequence shown in SEQ ID No: 13, and the light chain complementarily determining region LCDR3 comprises an amino acid sequence shown in SEQ ID No: 14; and

A-4. the heavy chain complementarity determining region HCDR1 comprises an amino acid sequence shown in SEQ ID No: 15, the heavy chain complementarily determining region HCDR2 comprises an amino acid sequence shown in SEQ ID No: 16, the heavy chain complementarity determining region HCDR3 comprises an amino acid sequence shown in SEQ ID No: 17, the light chain complementarily determining region LCDR1 comprises an amino acid sequence shown in SEQ ID No: 18, the light chain complementarity determining region LCDR2 comprises an amino acid sequence shown in SEQ ID No: 19, and the light chain complementarily determining region LCDR3 comprises an amino acid sequence shown in SEQ ID No: 20.

The anti-LAG-3 monoclonal antibody or the antigen binding fragment thereof provided by the present disclosure may specifically bind to LAG-3 and has a better binding activity. Blocking LAG-3 may reverse an inhibiting effect of LAG-3 on T cells, enhance the activity of the T cells, reduce the number of regulatory T cells, and also improve sensitivity of a T cell immune response, thereby may be used for treating immunological diseases or cancers.

Further, the monoclonal antibody or the antigen binding fragment thereof is a murine antibody molecule, and the murine antibody molecule is selected from any one of the following:
MA-1: the heavy chain variable region comprises an amino acid sequence shown in SEQ ID No: 21, and the light chain variable region comprises an amino acid sequence shown in SEQ ID No: 22;
MA-2: the heavy chain variable region comprises an amino acid sequence shown in SEQ ID No: 21, and the light chain variable region comprises an amino acid sequence shown in SEQ ID No: 23;
MA-3: the heavy chain variable region comprises an amino acid sequence shown in SEQ ID No: 24; and the light chain variable region comprises an amino acid sequence shown in SEQ ID No: 25; and
MA-4: the heavy chain variable region comprises an amino acid sequence shown in SEQ ID No: 26, and the light chain variable region comprises an amino acid sequence shown in SEQ ID No: 27; and
preferably, the murine antibody molecule is the MA-1.

In the present disclosure; mice are immunized with an LAG-3 antigen through mouse immunization, an immunization method is optimized, a phage display library is established; and the murine antibody molecule with a relatively high affinity, better activity and stability is screened out. A large number of cell-level experiments proved that compared with other 3 murine antibody molecules, MA-1 has a higher biological activity. Therefore, the MA-1 is preferably selected in the present disclosure.

Further, the murine antibody molecule further comprises a heavy chain constant region and a light chain constant region; the heavy chain constant region is one of murine IgG1 type, IgG2a type, IgG2b type, and IgG3 type, the heavy chain constant region of the IgG1 type has an amino acid sequence shown in SEQ ID No: 29, the heavy chain constant region of the IgG2a type has an amino acid sequence shown in SEQ ID No: 30, the heavy chain constant region of the IgG2b type has an amino acid sequence shown in SEQ ID No: 31 and the heavy chain constant region of the IgG3 type has an amino acid sequence shown in SEQ ID No. 32, and the light chain constant region a murine Cₖ chain with an amino acid sequence shown in SEQ ID No. 28; and

preferably, the heavy chain constant region is the murine IgG1 type.

Further, the monoclonal antibody or the antigen binding fragment thereof is a chimeric antibody molecule which comprises a heavy chain variable region of the murine antibody molecule, a light chain variable region of the murine antibody molecule, and a humanized antibody constant region; the humanized antibody constant region comprises a humanized antibody heavy chain constant region and a humanized antibody light chain constant region, the humanized antibody heavy chain constant region is one of human IgG1 type, IgG2 type, or IgG4 type, the heavy chain constant region of the IgG1 type has an amino acid sequence shown in SEQ ID No: 39, the heavy chain constant region of the IgG2 type has an amino acid sequence shown in SEQ ID No: 40, and the heavy chain constant region of the IgG4 type has an amino acid sequence shown in SEQ ID No: 41; and the humanized antibody light chain constant region is a human C, chain having an amino acid sequence shown in SEQ ID No: 42; and

preferably, the humanized antibody heavy chain constant region is the human IgG4 type.

The chimeric antibody molecule comprises the variable region sequence of the murine antibody molecule and the constant region of the human antibody, and the design of the chimeric antibody molecule is used to verify that the constant region of the present disclosure does not change the function of the CDR after humanization, which provides the basis for further research and development on the humanized antibody molecule.

Further, the monoclonal antibody or the antigen binding fragment thereof is a humanized antibody molecule, and the humanized antibody molecule is selected from any one of the following:
HA-1: the heavy chain variable region comprises an amino acid sequence shown in SEQ ID No: 33, and the light chain variable region comprises an amino acid sequence shown in SEQ ID No: 34,
HA-2: the heavy chain variable region comprises an amino acid sequence shown in SEQ ID No: 33, and the light chain variable region comprises an amino acid sequence shown in SEQ ID No: 35;
HA-3: the heavy chain variable region comprises an amino acid sequence shown in SEQ ID No: 36, and the light chain variable region comprises an amino acid sequence shown in SEQ ID No: 35;
HA-4: the heavy chain variable region comprises an amino acid sequence shown in SEQ ID No: 36, and the light chain variable region comprises an amino acid sequence shown in SEQ ID No: 34; and
HA-5: the heavy chain variable region comprises an amino acid sequence shown in SEQ ID No: 37, and the light chain variable region comprises an amino acid sequence shown in SEQ ID No: 38, and
preferably, the humanized antibody molecule is the HA-1.

According to the present disclosure, the humanized antibody molecule is obtained after humanization screening. In-vivo and in-vitro experimental verifications show that in the 5 humanized antibody molecules provided by the present disclosure, HA-1 has a relatively high activity and a most significant drug effect, such that HA-1 is preferably selected in the present disclosure.

Further, the humanized antibody molecule further comprises a heavy chain constant region and a light chain constant region, the heavy chain constant region is one of human IgG1 type, IgG2 type, or IgG4 type, the heavy chain constant region of the IgG1 type has an amino acid sequence shown in SEQ ID No: 39, the heavy chain constant region of the IgG2 type has an amino acid sequence shown in SEQ ID No: 40, the heavy chain constant region of the IgG4 type has an amino acid sequence shown in SEQ ID No. 41, and the light chain constant region is a human C, chain with an amino acid sequence shown in SEQ ID No: 42, and

preferably, the heavy chain constant region is the human IgG4 type.

Further, the humanized antibody molecule is a full-length antibody or an antibody fragment, the humanized antibody molecule comprises one or a combination of more of Fab, F(ab)2, Fv or ScFv.

The present disclosure further provides a polypeptide or a protein, comprising the anti-LAG-3 monoclonal antibody or the antigen binding fragment thereof.

The present disclosure further provides a polynucleotide sequence or combination: encoding an amino acid sequence of the anti-LAG-3 monoclonal antibody or the antigen-binding.

The present disclosure further provides a recombinant DNA expression vector, comprising the polynucleotide sequence or combination.

The present disclosure further provides a host cell transfected with the recombinant DNA expression vector, wherein the host cell comprises a prokaryotic cell, a yeast cell, an insect cell, or a mammalian cell, and preferably, the host cell is the mammalian cell, and the mammalian cell is an HEK293E cell, a CHO cell, or an NS0 cell.

The present disclosure further provides a drug or a pharmaceutical composition, comprising the anti-LAG-3 monoclonal antibody or the antigen binding fragment thereof.

The present disclosure further provides use of the anti-LAG-3 monoclonal antibody or the antigen binding fragment thereof in the preparation of a drug for treating cancers or immunological diseases;

preferably, the cancers comprises leukemia, lung cancer, stomach cancer, esophageal cancer, ovarian cancer, head and neck cancer, melanoma, kidney cancer, breast cancer, colorectal cancer, liver cancer, pancreatic cancer or bladder cancer; and

the immunological diseases comprises psoriasis, Crohn's disease, rheumatoid arthritis, primary biliary cirrhosis, systemic lupus erythematosus, multiple sclerosis, ulcerative colitis, and autoimmune hepatitis.

The present disclosure further provides use of the anti-LAG-3 monoclonal antibody or the antigen binding fragment thereof in combination with an anti-PD-1 monoclonal antibody in the preparation of a drug for treating a cancer or an immunological disease.

Further, the anti-PD-1 monoclonal antibody comprises DFPD1-9, DFPD1-10, DFPD1-11, DFPD1-12, DFPD1-13, nivolumab, pembrolizumab, toripalimab, sintilimab, tislelizumab, camrelizumab, penpulimab, or zimberelimab.

Preferably, the anti-PD-1 monoclonal antibody is DFPD1-10, and the DFPD1-10 comprises a light chain variable region shown in SEO ID No: 45 and a heavy chain variable region shown in SEC) ID No: 43.

Preferably, the anti-PD-1 monoclonal antibody is nivolumab.

Preferably, the cancer is selected from leukemia, king cancer, stomach cancer, esophageal cancer, ovarian cancer, head and neck cancer, melanoma, kidney cancer, breast cancer, colorectal cancer, liver cancer, pancreatic cancer or bladder cancer; and the immunological disease comprises psoriasis, Crohn's disease, rheumatoid arthritis, primary biliary cirrhosis, systemic lupus erythematosus, multiple sclerosis, ulcerative colitis, and autoimmune hepatitis.

The present disclosure has the following beneficial effects: the anti-LAG-3 monoclonal antibody or the antigen binding fragment thereof provided by the present disclosure may specifically bind to LAG-3, has a relatively high affinity and a very good biological activity, and may be used for treating various cancers or immunological diseases. The cancer includes but is not limited to leukemia, lung cancer, stomach cancer, esophageal cancer, ovarian cancer, head and neck cancer, melanoma, kidney cancer, breast cancer, colorectal cancer, liver cancer, pancreatic cancer or bladder cancer; and the immunological disease includes but is not limited to psoriasis, Crohn's disease, rheumatoid arthritis, primary biliary cirrhosis, systemic lupus erythematosus, multiple sclerosis, ulcerative colitis, and autoimmune hepatitis.

### BRIEF DESCRIPTION OF THE DRAWINGS

FIG. 1 is a plasmid profile of the pScFv-Disb-HS vector in Example 2 of the present disclosure;

FIG. 2 shows comparison of relative affinities of anti-LAG-3 phage monoclonal antibodies in the gradient-dilution ELISA in example 3 of the present disclosure;

FIG. 3 is a profile of a vector pTSE in Example 5 of the present disclosure;

FIG. 4 shows a denaturing polyacrylamide gel electrophoresis of murine antibody molecules in Example 5 of the present disclosure;

FIG. 5 shows comparison of binding abilities of murine antibodies to LAG-3 in Example 6 of the present disclosure;

FIG. 6 shows secretion of cytokine IL-2 by a murine antibody in a mixed lymphocyte reaction (MLR) in Example 7 of the present disclosure;

FIG. 7 shows a denaturing polyacrylamide gel electrophoresis of humanized antibody molecules in Example 12 of the present disclosure;

FIG. 8 shows comparison of binding abilities of humanized antibody molecules to LAG-3 in Example 15 of the present disclosure;

FIG. 9 shows activities of anti-LAG-3 humanized antibody molecules tested by an MLR in Example 16 of the present disclosure;

FIG. 10 shows a test effect of an anti-LAG-3 monoclonal antibody of Example 17 on inhibiting MC38 colorectal cancer in mice.

FIG. 11 shows an evaluation of molecular thermostability of an anti-LAG-3 monoclonal antibody, an HA-1 protein in Example 18 of the present disclosure;

FIG. 12 is a tumor volume growth curve graph of an anti-LAG-3 monoclonal antibody in combination with an anti-PD-1 monoclonal antibody in an MC38 colorectal cancer model in Example 22 of the present disclosure, and

FIG. 13 is a tumor weight column diagram of an anti-LAG-3 monoclonal antibody in combination with an anti-PD-1 monoclonal antibody in an MC38 colorectal cancer model in Example 22 of the present disclosure.

### DETAILED DESCRIPTION OF THE EMBODIMENTS

In order to understand the present disclosure more easily, before the examples are described, some technical and scientific terms of the present disclosure are firstly illustrated below:

The term “LAG-3” used herein refers to lymphocyte-activation gene 3. The LAG-3 herein includes but is not limited to diner-expressed LAG-3 (e.g., CD223 well-known in the art) on surfaces of activated T cells, NK cells, and B cells, and a soluble form of the LAG-3 found in human serum, all referred to herein as the LAG-3.

The term “antibody” used herein includes a whole antibody and any antigen binding fragment thereof. The antibody includes a murine antibody, a humanized antibody, a bispecific antibody or a chimeric antibody. Antibody may also be Fab, F(ab)2, Fv, or ScFv (single-chain antibody), and may be a naturally occurring antibody or also an altered (e.g., mutated, deleted, substituted, etc.) antibody.

The terms “variable region” and “constant region” used herein, i.e., a sequence region, near the N segments, of antibody heavy chain and light chain is a variable region (V region), and the rest amino acid sequence near the C segments is relatively stable, which is the constant region (C region). The variable region includes 3 complementarily determining regions (CDRs) and 4 framework regions (FRs), each light chain variable region and heavy chain variable region consists of 3 CDRs and 4 FRs, the 3 CDRs of a heavy chain are represented by HCDR1, HCDR2, and HCDR3, respectively, and the 3 CDRs of a light chain are represented by LCDR1, LCDR2, and LCDR3, respectively.

The term “murine antibody molecule” used herein is derived from an antibody obtained after immunization injection of a mouse with an LAG-3 antigen.

The term “chimeric antibody molecule” used herein refers to an antibody obtained by fusing the variable region of a murine antibody with the constant region of a humanized antibody, which may reduce the immune response induced by the murine antibody in a human body. The chimeric antibody is prepared by inserting light and heavy chain variable region genes of a murine monoclonal antibody into expression vectors containing humanized antibody constant regions, so that the variable regions of the light chain and the heavy chain in the expressed antibody molecule are murine, while the constant region is humanized, and near 2/3 part of the whole antibody molecule is humanized. The antibodies thus generated reduce immunogenicity of the murine antibody and at the same time retains the ability of the parental antibody to specifically bind to the antigen.

The term “humanized antibody molecule” used herein refers to grating the CDRs of the murine monoclonal antibody to the variable regions of the humanized antibody to replace the CDRs of the humanized antibody, such that the humanized antibody may acquire an antigen binding specificity of the murine monoclonal antibody while reducing its heterogeneity.

The term “CHO cell” is a Chinese hamster ovary cell; the term “HEK 233E cell” refers to the human embryonic kidney 293E cell; and the term “NS0 cell” is a mouse NS0 thymoma cell.

The present disclosure is described in further detail below with reference to the examples.

### EXAMPLE 1

Example 1 of the present disclosure provided an anti-LAG-3 monoclonal antibody or an antigen binding fragment thereof, specifically comprising a heavy chain variable region and a light chain variable region, wherein the heavy chain variable region comprised 3 heavy chain complementarity determining regions, respectively represented by HCDR1, HCDR2, and HCDR3, and the light chain variable region comprised 3 light chain complementarity determining regions, respectively represented by LCDR1. LCDR2, and LCDR3, and the monoclonal antibody or the antigen binding fragment thereof was selected from any one of the fallowing:

| Combination | HCDR1 | HCDR2 | HCDR3 | LCDR1 | LCDR2 | LCDR3 |
|---|---|---|---|---|---|---|
| A-1 | DTYMH | KIDPANGNTKYDPKFQG | DTTVGLDY | RASSSVNYMY | YTSKLAS | QQWSSNPHT |
| | (SEQ ID | (SEQ ID No: 2) | (SEQ ID | (SEQ ID | (SEQ ID | (SEQ ID |
| | No: 1) | | No: 3) | No: 4) | No: 5) | No: 6) |
| A-2 | DTYMH | KIDPANGNTKYDPKFQG | DTTVGLDY | RASSSVNYMY | TYSNLAP | QQFTSSPSMT |
| | (SEQ ID | (SEQ ID No: 2) | (SEQ ID | (SEQ ID | (SEQ ID | (SEQ ID |
| | No: 1) | | No: 3) | No: 4) | No: 7) | No: 8) |
| A-3 | GYSFTSY | WIFPGTGNTRYNEKFKG | IGGRLTGDAMDY | RASESVDSYGNSFMH | LASNLES | QQNNEDPWT |
| | (SEQ ID | (SEQ ID No: 10) | (SEQ ID | (SEQ ID | (SEQ ID | (SEQ ID |
| | No: 9) | | No: 11) | No: 12) | No: 13) | No: 14) |
| A-4 | TYAVH | VIWSGGSTDYNIAFISR | LDGTFFDY | TASSSVSSSYLH | STSNLAS | HQYHRSPPT |
| | (SEQ ID | (SEQ ID No: 16) | (SEQ ID | (SEQ ID No: 18) | (SEQ ID | (SEQ ID |
| | No: 15) | | No: 17) | | No: 19) | No: 20) |

### EXAMPLE 2 Screening of Murine Antibody Molecule

According to the present disclosure, mice were immunized with an LAG-3 antigen, an immunization method was optimized, a phage display library was established, and construction, screening, and identification of the specific phage display library were as follows:

Step 1: immunization of mice with LAG-3 antigen

1. Experimental animals:
species and strains: female BALB/c mice;
weight: 18-20 g; and

Experimental animal provider: Beijing HFK Bioscience Co., Ltd.

2. Immunization: the mice were immunized, and the immune antigen was human LAG-3 (the gene was synthesized by GenScript Biotech Corporation (Nanjing), a vector was constructed, and the protein was expressed and purified by our company).

Step 2: construction of aphage antibody library

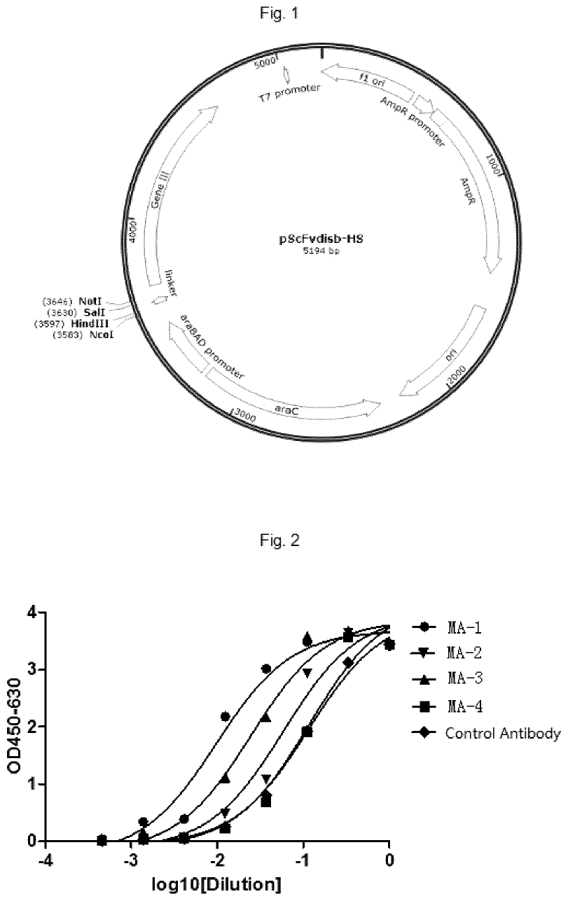

Mouse splenocytes with a higher titer was taken, total RNA in the mouse splenocytes was extracted with a Trizol reagent (purchased from Ambion with the Cat. No. 15596026), cDNA was obtained by RT PCR, a PCR amplification was performed with cDNA as a template and degenerate primers (the used degenerate primers refer to a reference. Journal of Immunological Methods, 233(2000), 167-177), thus a heavy chain variable region gene library (VH) and a light chain variable region gene library (VL) of the immunized mouse antibody were obtained, a light chain and a heavy chain were double-enzyme-digested respectively, and connected to a vector which was also enzyme digested step-by-step to construct a pScFv-Disb-HS-VH-VL gene library, a PscFv-DisB-HS vector was modified from a pComb3 vector (purchased from the Biovector Science Lab, Inc.) with a series of gene cloning methods, such that the vector was used for constructing and expressing a phage single-chain antibody library. The modified vector was named pScFv-Disb-HS vector, its obtained plasmid profile was shown in FIG. 1, and a mouse immune phage antibody library was constructed on the basis of the vector.

Step 3: an immune tube was coated with LAG-3 as an antigen, an amount of the antigen coating is 5 μg/500 μL/tube, and coating was performed at CC overnight, the immune tube and the immune phage antibody library were blocked at room temperature for 1 h with 4% skim milk powder/PBST respectively. The blocked immune phage antibody library was added into the immune tube with an input amount of phages of about 10⁵-10¹² for an antigen-antibody binding, alter reacting at room temperature for 1 h, unbound phages were washed away with PBST-PBS, eluting was performed with 0.1 M of glycine-HCl at a pH of 2.2, and finally an eluted phage antibody solution was neutralized to a pH of 7.0 with 1.5M of Iris-HCl at a pH of 8.8.

Step IV: 10 ml of a TG 1 bacterial solution growing to a logarithmic phase was infected with the neutralized phages, let the solution stood in an incubator at 37° C. for 30 min, a part of the bacterial solution was taken out for gradient dilution, and coated on a 2YTAG plate for the calculation of the yield of the phages. The remaining bacterial solution was centrifuged, a supernatant was discarded, and the bacterial precipitate was resuspended in a small amount of a culture medium, aspirated, and coated on a 2YTAG large plate in preparation for a next round of screening.

Step V: the infected plated bacteria were scraped from the large plate, inoculated to a 2YTAG liquid culture medium, shaken to a logarithmic phase, an M13KO7 helper phage was added for superinfection, the bacteria were cultured overnight at 220 rpm at 28° C. to prepare the phages, and the phages were precipitated and purified by PEG/NaCl for a next round of screening to perform a round of enrichment screening of the phage library.

Step VI: screening of LAG-3 phage single-chain antibody positive clone: after a round of screening; a well-separated monoclonal colonies was picked, inoculated into a 96-deep-well plate containing a 2YTAG liquid culture medium; cultured at 37° C. under the condition of 220 rpm to a logarithmic growth phase, about 10¹⁰ of the helper phages M13KO7 were added per well, and static infection was performed at 37° C. for 30 min. Centrifugation was performed at 4,000 rpm for 15 min, a supernatant was discarded, the bacterial precipitate was resuspended with 2YTAK, and the bacteria were cultured overnight at 28° C. under the condition of 220 rpm. After centrifugation at 4,000 rpm and 4° C. for 15 min, an amplified phage supernatant was sucked for an ELISA identification, finally four anti-LAG-3 murine antibody molecules with a relatively high affinity were screened, named as MA-1, MA-2, MA-3, and MA-4 respectively, the obtained monoclonal antibodies were subjected to gene sequencing to be determined as correct antibody sequences, and after sequencing, the sequences of the 4 monoclonal antibodies screened above were as follows:

| Murine antibody | Heavy chain variable | Light chain variable |
|---|---|---|
| molecule | region sequence | region sequence |
| MA-1 | SEQ ID No: 21 | SEQ ID No: 22 |
| MA-2 | SEQ ID No: 21 | SEQ ID No: 23 |
| MA-3 | SEQ ID No: 24 | SEQ ID No: 25 |
| MA-4 | SEQ ID No: 26 | SEQ ID No: 27 |

Specifically, SEQ ID No: 21 (amino acid sequence
of heavy chain variable region of MA-1 and MA-2):
EVQLQQSGAELVKPGASVKLSCTASGFNIKDTYMHWVKQRPEQGLEWIG
KIDPANGNTKYDPKFQGKATITADTSSNTAYLQLSRLTSEDTAVYYCAR
TTVGLDYWGQGTTLVTVSS;
(amino acid sequence of light chain variable
region of MA-1):
SEQ ID No: 22
DIVITQSTAIMSASLGEKVTMSCRASSSVNYMYWYQQKSDASPKLWIYY
TSKLASGVPARFSGSGSGTSYSLTISSMEAEDAATYYCQQWSSNPHTFG
GGTKLEIK;
(amino acid sequence of light chain variable
region of MA-2):
SEQ ID No: 23
DIVLTQTPAIMSASLGEKVTMSCRASSSVNYMYWYQQKSDASPKLWIYY
TSNLAPGVPARFSGSGSGNSYSLTISSMEGEDAATYYCQQFTSSPSMTF
GGGTKLEIK;
(amino acid sequence of heavy chain variable
region of MA-3):
SEQ ID No: 24
EVKLEESGPELVKPGASVKISCKASGYSFTSYYIHWVKQRPGQGLEWIG
WIFPGTGNTRYNEKFKGKATLTADTSSSTVYMQLSGLTSEDSAVYFCAR
IGGRLTGDAMDYWGQGTSVTVSS;
(amino acid sequence of light chain variable
region of MA-3):
SEQ ID No: 25
DIVLTQSTASLAVSLGQRATISCRASESVDSYGNSFMHWYQQKPGQPPK
LLIYLASNLESGVPARFSGSGRTDFTLTIDPVEADDAATYYCQQNNEDP
WTFGGGTKLEIK;
(amino acid sequence of heavy chain variable
region of MA-4):
SEQ ID No: 26
QVKLEESGPGLVQPSQSLSITCTVSAFSLTTYAVHWVRQSPGKGLEWLG
VIWSGGSTDYNTAFISRLNITKDNSKSQVFFKMNSLQANDTAIYYCARL
DGTFFDYWGQGTTLTVSS;
and
(amino acid sequence of light chain variable
region of MA-4):
SEQ ID No: 27
DIVLTQTPAIMSASLGERVTMTCTASSSVSSSYLHWYQQKPGSSPKLWI
YSTSNLASGVPARFSGSGSGTSYSLTISSMEAEDAATYYCHQYHRSPPT
FGGGTKLEIK.

### EXAMPLE 3 Comparison of affinities of anti-LAG-3 phage monoclonal antibodies by gradient-dilution

The 4 murine antibody molecules (MA-1, MA-2, MA-3, and MA-4) obtained in example 2 were subjected to display and purification of monoclonal phages, then the phages were subjected to a gradient-dilution ELISA experiment to identify an affinity, and an anti-LAG-3 monoclonal antibody provided in patent application CN105992595A was selected as a control antibody. The specific method was as follows:

100 ng/well/100 μl of LAG-3 was coated with a carbonate buffer solution at a pH of 9.6 overnight at 4° C. and washed three times with PBST, the 4 phage monoclonal antibodies screened in example 2 were respectively diluted with PBST in a three-fold gradient, and 1000 of the diluted sample was added to each well and stood at room temperature for 1 hour. An EUSA plate was washed with PBST, and an HRP-anti-M13 monoclonal antibody diluted with the PBST was added to the EUSA plate and stood at room temperature for 1 h. Color development was performed with a TMB color developing kit at room temperature for 10 minutes, and terminated with 2M of H₂SO₄. Reading was performed at 450 nm/630 nm, a corresponding EC50 value was calculated, and specific data was as follows:

| | | | | | Control |
|---|---|---|---|---|---|
| Clone | MA-1 | MA-2 | MA-3 | MA-4 | antibody |
| EC50 | 0.010 | 0.058 | 0.024 | 0.117 | 0.115 |

Based on the above data and FIG. 2, the 4 different murine antibody molecules screened in example 2 may all bind to LAG-3. The monoclonal antibodies provided by the present disclosure had a relatively high affinity to the LAG-3.

### EXAMPLE 4

On the basis of example 2, example 4 of the present disclosure further defined that the murine antibody molecule further comprised a heavy chain constant region and a light chain constant region, wherein the heavy chain constant region was one of murine IgG1 type, IgG2a type, IgG2b type, and IgG3 type, the heavy chain constant region of the IgG1 type had an amino acid sequence shown in SEQ ID No: 29, the heavy chain constant region of the IgG2a type had an amino acid sequence shown in SEQ ID No: 30, the heavy chain constant region of the IgG2b type had an amino acid sequence shown in SEQ ID No: 31, and the heavy chain constant region of the IgG3 type had an amino acid sequence shown in SEQ ID No: 32: and the light chain constant region was a murine Cₖ chain with an amino acid sequence shown in SEQ ID No: 28 The specific sequences were as follows:

(light chain constant region sequence of murine
Cₖ chain):
SEQ ID No: 28
ADAAPTVSIFPPSSEQLTSGGASVVCFLNNFYPKDINVKWKIDGSERQNG
VLNSWTDQDSKDSTYSMSSTLTLTKDEYERHNSYT
(heavy chain constant region sequence of murine
IgGI type):
SEQ ID No: 29
AKTTPPSVYPLAPGSAAQTNSMVTLGCLVKGYFPEPVTVTWNSGSLSSGV
HTFPAVLQSDLYTLSSSVTVPSSTWPSETVTCNVAHPASSTKVDKKIVPR
DCGCKPCICTVPEVSSVFIFPPKPKDVLTITLTPKVTCVVVDISKDDPEV
QFSWFVDDVEVHTAQTQPREEQFNSTFRSVSELPIMHQDWLNGKEFKCRV
NSAAFPAPIEKTISKTKGRPKAPQVYTIPPPKEQMAKDKVSLTCMITDFF
PEDITVEWQWNGQPAENYKNTQPIMDTDGSYFVYSKLNVQKSNEWAGNTF
TCSVLHEGLHNHHTEKSLSHSPG;
(heavy chain constant region sequence of murine
IgG2a type):
SEQ ID No: 30
AKTTAPSVYPLAPVCGDTTGSSVTLGCLVKGYFPEPVTLTWNSGSLSSGV
HTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVS
EDDPDVQISWFVNNVEVHTQAQTQTHREDYNSTLRVVSALPIQHQDWMSG
KEFKCKVNNKDLPAPIERTISKPKGSVRAPQVYVLPPPEEEMTKKQVTLT
CMVTDFMPEDIYVEWTNNGKTELNYKNTEPVLDSDGSYFMYSKLRVEKKN
WVERNSYSCSVVHEGLHNHHTTKSFSRTPGK;
(heavy chain constant region sequence of murine
IgG2b type):
SEQ ID No: 31
AKTTPPSVYPLAPGCGDTTGSSVTLGCLVKGYFPESVTVTWNSGSLSSSV
HTFPALLQSGLYTMSSSVTVPSSTWPSQTVTCSVAHPASSTTVDKKLEPS
GPISTINPCPPCKECHKCPAPNLEGGPSVFIFPPNIKDVLMISLTPKVTC
VVVDVSEDDPDVQISWFVNNVEVHTAQTQTHREDYNSTIRVVSTLPIQHQ
DWMSGKEFKCKVNNKDLPSPIERTISKIKGLVRAPQVYILPPPAEQLSRK
DVSLTCLVVGFNPGDISVEWTSNGHTEENYKDTAPVLDSDGSYFIYSKLN
MKTSKWEKTDSFSCNVRHEGLKNYYLKKTISRSPGK;
and
(heavy chain constant region sequence of murine
IgG3 type):
SEQ ID No: 32
ATTTAPSVYPLVPGCSDTSGSSVTLGCLVKGYFPEPVTVKWNYGALSSGV
RTVSSVLQSGFYSLSSLVTVPSSTWPSQTVICNVAHPASKKTELIKRIEP
RIPKPSTPPGSSCPPGNILGGPSVFIFPPKPDKALMISLTPKVTCVVVDV
SEDDPDVHVSWFVDNKEVHTAWTQPREAQYNSTFRVVSALPIQHQDWMRG
KEFKCKVNNKALPAPIERTISKPKGRAQTPQVYTIPPPREQMSKKKVSLT
CLVTNFFSEAISVEWERNGELEQDYKNTPPILDSDGTYFLYSKLTVDTDS
WLQGEIFTCSVVHEALHNHHTQKNLSRSPELELNETCAEAQDGELDGLWT
TITIFISLFLLSVCYSASVTLFVKWIFSSVVQVKQTAIPDYRNMIGQGA.

### EXAMPLE 6 Preparation of anti-LAG-3 Murine Antibody Molecule

On the basis of example 4, example 5 of the present disclosure preferably defined that the heavy chain constant region of the murine antibody molecule was a murine IgG1 type, and the IgG1 type comprised the amino acid sequence shown in SEQ ID No: 29; and the light chain constant region was a murine chain, and the murine Ci chain comprised an amino acid sequence shown in SEQ ID No: 28. A method for preparing the antibody specifically comprised the following steps.

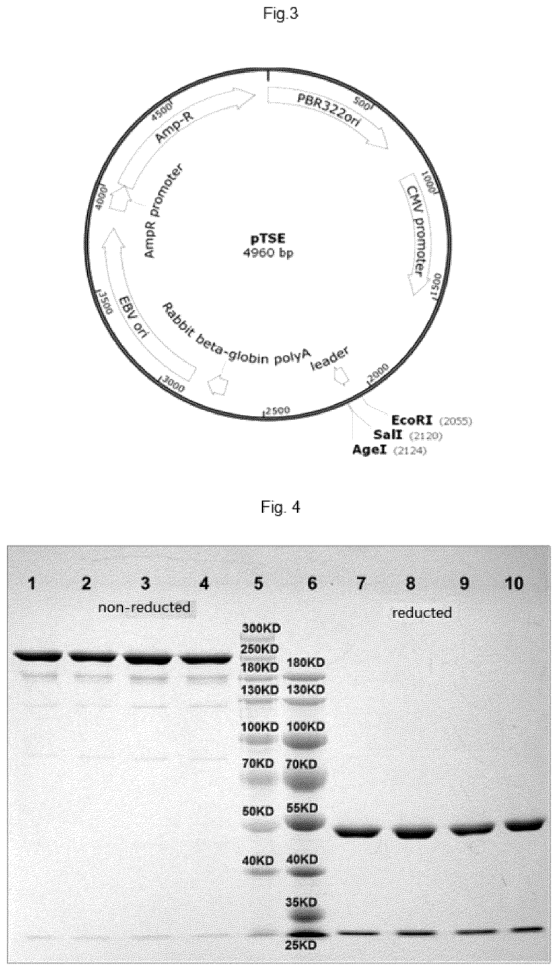

1. Encoding genes of heavy chain VH and light chain VL of the 4 monoclonal antibodies screened in example 2 were respectively cloned into a pTSE vector (see FIG. 3) containing heavy chain and light chain constant region genes, preferably, a heavy chain constant region was the marine IgG1 type constant region (amino acid sequence shown in SEQ ID No: 29), a light chain constant region was the murine Cₖ chain (amino acid sequence shown in SEQ ID No: 28), and the structure of the pTSE vector was shown in FIG. 3 (a preparation process of the pTSE vector referred to page 3, paragraph [0019] of the description of CN103525868A).

2 The vector was transiently transfected into HEK293E cells (purchased from the Institute of Basic Medicine of Chinese Academy of Medical Sciences, Cat. No: GNHu43) for expressing an antibody, 4 monoclonal antibodies were obtained by a protein A affinity column purification using an AKTA instrument, a protein concentration was measured with a BCA kit (purchased from Beijing HuitianDongfang Technology Co., Ltd., Cat. No: BCA0020), and then a protein size was identified by SDS-PAGE. The results were shown in FIG. 4, non-reduced MA-1, MA-2, MA-3 and MA-4, protein molecular weight Marked and Marker2, and reduced MA-1, MA-2, MA-3 and MA-4 murine anti-LAG-3 monoclonal antibodies were sequentially present from left to right, and the molecular weight of each band was consistent with a theoretical value.

### EXAMPLE 6 Binding experiment of murine antibody to LAG-3

100 ng/well/100 μl of LAG-3 was coated with a carbonate buffer solution at a pH of 9.6 overnight at 4° C. The LAG-3 was washed five times with 300 μl/well of PBST, then 1% of BSA-PBST was added, the LAG-3 was blocked at 37° C. for 1 h, MA-1, MA-2, MA-3 and MA-4 murine antibodies with different dilution concentrations were added, wherein the 4 whole antibodies all had an initial highest concentration of 5 μg/ml and respectively diluted by 3 times, and 12 gradients were made for each antibody, and incubated at 37° C. for 1 h. The obtained product was washed five times with 300 μl/well of PBST, then Anti-Mouse Fc-HRP diluted with 1% BSA-PBST at 1:10,000 was added, and the mixture was incubated at 37° C. for 1 h Color development was performed with 100 μl/well of a TMB color developing kit at room temperature for 8 minutes, and terminated with 2M of H₂SO₄. Reading was performed at 450 nm/630 nm, a corresponding EC50 value was calculated, and specific data was as follows:

| Clone | MA-1 | MA-2 | MA-3 | MA-4 |
|---|---|---|---|---|
| EC50 (ng/ml) | 10.08 | 25.99 | 255.9 | 22.12 |

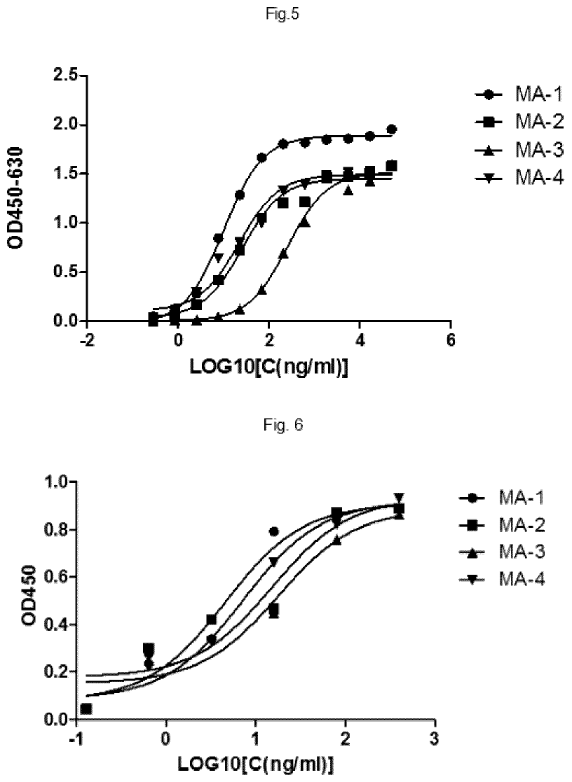

Based on the above data and FIG. 5, 4 different screened murine antibodies may all bind to LAG-3. In addition, the EC50 value of the MA-1 in the 4 murine antibody molecules was lowest. This indicated that the MA-1 had a best binding ability and highest affinity.

### EXAMPLE 7 Effect of murine antibodies on secretion amount of cytokine IL-2 in mixed lymphocyte reaction

Fresh peripheral blood mononuclear cells (PBMCs) were separated by a density gradient centrifugation and CD14′ T cells were sorted by magnetic beads; and the CD14′ T cells were cultured by a culture medium of 20 ng/mL of GM-CSF and 10 ng/mL of IL-4, the culture medium was changed every 2 days, and the cells were induced into dendritic cells (DCs) in 7-10 days. Two days before the DCs were used, 25 ng/mL of TNF-α was added to induce the DCs into mature DCs, and the mature DCs were collected and prepared into a cell suspension with a cell density of 1×10⁵ cells/mL. The CDC⁴⁺ T cells were sorted from the PBMCs with magnetic beads, counted, and prepared into a cell suspension with a cell density of 1×10⁶ cells/mL. 100 μl of each of the CDC T cells and the DCs were taken and added into a 96-well plate according to a ratio of 10:1.

The MA-1, MA-2, MA-3 and MA-4 murine antibodies prepared in example 5 were respectively subjected to a 4-fold gradient dilution, 6 gradients were set for each antibody, 500 of each antibody was respectively taken and added into a 96-well plate, a concentration of IL-2 after culturing for 5 days was detected, reading was performed at 450 nm, a corresponding EC50 value was calculated, and specific data was as follows:

| Clone | MA-1 | MA-2 | MA-3 | MA-4 |
|---|---|---|---|---|
| EC50 (μg/ml) | 0.024 | 0.030 | 0.076 | 0.121 |

Based on the data and FIG. 6, the 4 murine antibody molecules screened by the present disclosure all had a relatively good activity. In addition, it may further be obtained from the data that the EC50 value of the MA-1 was the lowest among the 4 monoclonal antibodies screened by the present disclosure, and thus the MA-1 had a best activity. Therefore, in the present disclosure the murine antibody molecule MA-1 may be humanized.

### EXAMPLE 8

Example 8 of the present disclosure further defined that the monoclonal antibody or the antigen binding fragment thereof was a chimeric antibody molecule which comprised a heavy chain variable region of the murine antibody molecule, a light chain variable region of the murine antibody molecule, and a humanized antibody constant region in example 2; the humanized antibody constant region comprised a humanized antibody heavy chain constant region and a humanized antibody light chain constant region, the humanized antibody heavy chain constant region was one of human IgG1 type, IgG2 type, or IgG4 type, the heavy chain constant region of the IgG1 type had an amino acid sequence shown in SEQ ID No: 39, the heavy chain constant region of the IgG2 type had an amino acid sequence shown in SEQ ID No: 40, and the heavy chain constant region of the IgG4 type had an amino acid sequence shown in SEQ ID No: 41; and the humanized antibody light chain constant region was a human Cₖ chain having an amino acid sequence shown in SEQ ID No: 42.

(amino acid sequence of heavy chain constant
region of human IgGI type):
SEQ ID No: 39
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSCVMHEALHNHYTQKSLSLSPGK;
(amino acid sequence of heavy chain constant
region of human IgG2 type):
SEQ ID No: 40
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKS
RWQQGNVFSCSVMHEALHNHYTQKSLSLSPGK;
(amino acid sequence of heavy chain constant
region of human IgG4 type):
SEQ ID No: 41
ASTKGPSVFLPAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHELAHNHYTQKSLSLSLGK;
and
(amino acid sequence of light chain constant
region of human & chain):
SEQ ID No: 42
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

### EXAMPLE 9 Preparation of Chimeric Antibody Molecule

On basis of example 8. example 9 of the present disclosure further defined that a heavy chain constant region of a chimeric antibody molecule was a human IgG4 type, and the heavy chain constant region of the IgG4 type had an amino acid sequence shown in SEQ ID No: 41 and a light chain constant region of the chimeric antibody was the human Cₖ chain with an amino acid sequence shown in SEQ ID No: 42.

A specific preparation method was as follows:

The genes of the heavy chain variable region VH (SEQ ID No: 21) and light chain variable region VL (SEQ ID NO: 22) of the antibody molecule MA-1 screened from the phage antibody library of example 2 kept unchanged murine sequences, and were cloned into the pTSE vector (shown in FIG. 3) containing heavy chain and light chain constant region genes respectively, the heavy chain constant region was human IgG4 type (amino acid sequence shown in SEQ ID No: 41) and the light chain constant region was human Ct chain (amino acid sequence shown in SEQ ID No: 42). The vector was transiently transfected into HEK293E cells (purchased from the Institute of Basic Medicine of Chinese Academy of Medical Sciences, Cat. No:GNHu43) for expressing an antibody to obtain a chimeric antibody CA-1.

### EXAMPLE 10 Humanization of Murine Antibody Molecule MA-1

First, the sequence of the murine antibody molecule MA-1 in example 2 was compared with the human antibody germline database (v-base), human antibody light chain and heavy chain germlines with a relatively high homology were searched as candidate sequences, and then sequences of CDRs of the murine antibody molecule MA-1 were grafted onto a humanized candidate sequence for a homologous modeling. Then, the key framework ammo acid residues that may play an important role in maintaining the CDR loop structure were calculated by three-dimensional structure simulation, so as to design the back mutation of the humanized antibody.

The designed humanized variable regions containing the back mutation of light chain and heavy chain were optimized and synthesized by GenScript Biotech Corporation (Nanjing), and then connected to a transient expression vector. The combination of the light chain and the heavy chain obtained by humanization was analyzed to obtain the following humanized antibody molecules: HA1, HA-2, HA-3, HA-4, and HA-5, wherein the sequences of the screened 5 monoclonal antibodies were as follows:

| Monoclonal | Heavy chain | Light chain |
|---|---|---|
| antibody | variable region | variable region |
| HA-1 | SEQ ID No: 33 | SEQ ID No: 34 |
| HA-2 | SEQ ID No: 33 | SEQ ID No: 35 |
| HA-3 | SEQ ID No: 36 | SEQ ID No: 35 |
| HA-4 | SEQ ID No: 36 | SEQ ID No: 34 |
| HA-5 | SEQ ID No: 37 | SEQ ID No: 38 |

Specifically, SEQ ID No: 33 (amino acid sequence
of heavy chain variable region of HA-1 and HA-2):
QVQLVQSGAEVKKPGASVKVSCKASGYTFTDTYMHWVRQAPGQGLEWIG
KIDPANGNTKYDPKFQGRATITADTSISTAYMELSRLRSDDTAVYYCAR
DTTVGLDYWGQGTLVTVSS;
SEQ ID No: 34 (amino acid sequence of light
chain variable region of HA-1 and HA-4):
DIVITQSPAFLSVTPGEKVTITCRASSSVNYMYWYQQKPDASKPLWIYY
TSKLASGVPSRFSGSGSGTDYTFTISSLEAEDAATYYCQQWSSNPHTFG
GGTKVEIK;
SEQ ID No: 35 (amino acid sequence of light
chain variable region of HA-2 and HA-3):
DIVITQSPAFLSVTPGEKVTITCRASSSVNYMYWYQQKPDQAPKLLIYY
TSKLASGVPSRFSGSGSGTDYTFTISSLEAEDAATYYCQQWSSNPHTFG
GGTKVEIK;
SEQ ID No: 36 (amino acid sequence of heavy
chain variable region of HA-3 and HA-4):
QVQLVQSGAEVKKPGASVKVSCKASGYNITDTYMHWVRQAPGQGLEWIG
KIDPANGNTKYDPKFQGRATITADTSISTAYMELSRLRSDDTAVYYCAR
DTTVGLDYWGQGTLVTVSS;
SEQ ID No: 37 (amino acid sequence of heavy
chain variable region of HA-5):
QVQLVQSGAEVKKPGASVKVSCKASGYNIKDTYMHWVKQAPGQGLEWIG
KIDPANGNTKYDPKFQGKATITADTSISTAYMELSRLRSDDTAVYYCAR
DTTVGLDYWGQGTLVTVSS;
and
SEQ ID No: 38 (amino acid sequence of light
chain variable region of HA-5):
DIVITQSPAFLSVTPGEKVTITCRASSSVNYMYWYQQKPDASPKLWIYY
TSKLASGVPARFSGSGSGTYTFTISSLEAEDAATYYCQQWSSNPHTFGG
GTKVEIK.

### EXAMPLE 11

On the basis of example 10, example 11 of the present disclosure further defined that the humanized antibody molecule further comprised a heavy chain constant region and a light chain constant region, the heavy chain constant region was one of human IgG1 type, IgG2 type, or IgG4 type, the heavy chain constant region of the IgG1 type had an amino acid sequence shown in SEQ ID No: 39, the heavy chain constant region of the IgG2 type had an amino acid sequence shown in SEQ ID No: 40, the heavy chain constant region of the IgG4 type had an amino acid sequence shown in SEQ ID No: 41 and the light chain constant region was a human C chain with an amino acid sequence shown in SEQ ID No: 42.

### EXAMPLE 12 Preparation of Humanized Antibody Molecule

On basis of example 11, example 12 of the present disclosure further defined that the heavy chain constant region of the humanized antibody molecule was human IgG4 type, and the heavy chain constant region of the IgG4 type had an amino acid sequence shown in SEQ ID No. 41; and a light chain constant region was the human Cₖ chain with an amino acid sequence shown in SEQ ID No 42.

Encoding genes of heavy chain VH and light chain VL of the 5 humanized antibody molecules obtained by humanization in example 10 were cloned into a pTSE vector (see FIG. 3) containing heavy chain and light chain constant region genes respectively, a heavy chain constant region was the human IgG4 type (amino acid sequence shown in SEQ ID No: 41), and a light chain constant region was the Cₖ chain (amino acid sequence shown in SEC) ID No: 42)

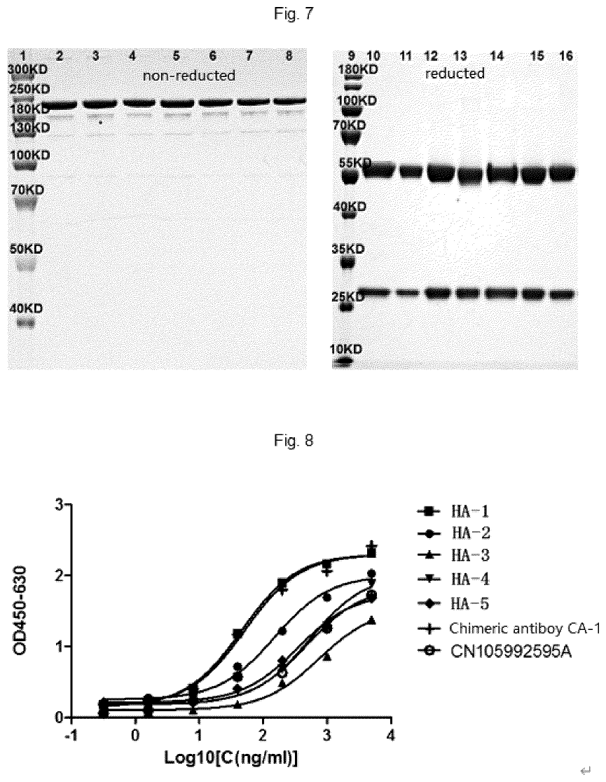

The chimeric antibody CA-1 and the humanized antibody were transiently transfected into HEK293E cells (purchased from the Institute of Basic Medicine of Chinese Academy of Medical Sciences, Cat. No. GNHu43) for expressing an antibody, monoclonal antibodies were obtained by a protein A affinity column purification using an AKTA instrument, a protein concentration was measured with a BCA kit (purchased from Beijing HuitianDongfang Technology Co., Ltd., Cat. No. BCA0020), and then a protein size was identified by SDS-PAGE. Results were shown in FIG. 7, non-reduced protein molecular weight Marker 1, HA-1, HA-2, HA-3, HA-4, HA-5; chimeric antibody CA-1 prepared in example 9, and anti-LAG-3 monoclonal antibody provided in core patent application CN105992595A, and reduced protein molecular weight Marker 2, HA-1, HA-2, HA-3, HA-4, HA-5, chimeric antibody CA-1; and anti-LAG-3 monoclonal antibody provided in core patent application CN105992595A were sequentially present from left to right, and the molecular weight of each band was consistent with a theoretical value.

### EXAMPLE 13

On the basis of the above example, example 13 of the present disclosure further defined that the humanized antibody molecule was a full-length antibody or an antibody fragment, and comprised one or a combination of more of Fab, F(ab)2, Fv or ScFv.

### EXAMPLE 14

On the basis of the above example, example 14 of the present disclosure further defined the following solution.

A polypeptide or a protein comprising the anti-LAG-3 monoclonal antibody or the antigen binding fragment thereof defined by any one of the above examples was further defined.

A polynucleotide sequence or combination encoding an amino acid sequence of the anti-LAG-3 monoclonal antibody or the antigen binding fragment thereof defined by any one of the above examples was further defined.

A recombinant DNA expression vector comprising the polynucleotide sequence or combination was further defined.

A host cell transfected with the defined recombinant DNA expression vector was further defined, wherein the cell comprised a prokaryotic cell, a yeast cell, an insect cell, or a mammalian cell; and

preferably, the host cell was the mammalian cell, and the mammalian cell was an HEK293E cell, a CHO cell, or an NS0 cell.

A drug or pharmaceutical composition comprising the anti-LAG-3 monoclonal antibody or the antigen-binding defined by any one of the above examples was further defined.

The present disclosure further provided use of the anti-LAG-3 monoclonal antibody or the antigen binding fragment thereof in the preparation of a drug for treating a cancer or an immunological disease; and

preferably, the cancer included but was not limited to leukemia, lung cancer, stomach cancer, esophageal cancer, ovarian cancer, head and neck cancer, melanoma, kidney cancer, breast cancer, colorectal cancer, liver cancer, pancreatic cancer or bladder cancer; and the immunological disease included but was not limited to psoriasis; Crohn's disease, rheumatoid arthritis, primary biliary cirrhosis, systemic lupus erythematosus, multiple sclerosis, ulcerative colitis, and autoimmune hepatitis.

### EXAMPLE 10 Binding Experiment of Humanized Antibody Molecule to LAG-3

100 ng/well/100 μl of LAG-3 was coated with a carbonate buffer solution at a pH of 9.6 overnight at 4° C. The LAG-3 was washed five times with 300 μl/well of PBST, then 1% of BSA-PBST was added, the LAG-3 was blocked at 37° C. for 2 h, the humanized antibodies of HA-1, HA-2, HA-3, HA-4, and HA-5, the chimeric antibody CA-1 prepared in example 9, and the anti-LAG-3 antibody in patent application CN105992595A with different dilution concentrations were added, wherein the 7 antibodies all had an initial highest concentration of 5 μg/ml and respectively diluted by 5 times, and each antibody had 8 gradients, and the mixture was incubated at 37° C. for 1 h The obtained product was washed five times with 300 μl/well of PBST, then Anti-Human Fc-HRP diluted with 1% BSA-PBST at 1:10,000 was added; and the mixture was incubated at 37° C. for 1 h. Color development was performed with 100 μl/well of a TMB color developing kit at room temperature for 8 minutes, and terminated with 2M of H₂SO₄. Reading was performed at 450 nm/630 nm, a corresponding EC50 value was calculated, and specific data was as follows:

| | | | | | | Chimeric | CN- |
|---|---|---|---|---|---|---|---|
| Clone | HA-1 | HA-2 | HA-3 | HA-4 | HA-5 | antibody CA-1 | 105992595A |
| EC50 | 46.25 | 150.7 | 102.5 | 538.8 | 350.3 | 52.16 | 747.2 |
| (ng/ml) | | | | | | | |

Based on the data and experiment result shown in FIG. 8, the 5 different humanized antibody molecules may all bind to LAG-3. The EC50 values of the 5 different monoclonal antibodies provided by the present disclosure were all significantly lower than that of the anti-LAG-3 antibody in patent application CN105992595A, which indicated that the monoclonal antibodies provided by the present disclosure had a strong binding ability to the LAG-3 and a high affinity. Besides, it may also be seen from FIG. 8 and the data that the EC50 value of the HA-1 among the 5 different monoclonal antibodies was the lowest, which indicated that the monoclonal antibody has a best binding ability to the LAG-3 and a highest affinity. Meanwhile, the EC50 value of the HA-1 was similar to that of the chimeric antibody CA-1; which indicated that the humanized HA-1 retained the high affinity of the murine parent antibody MA-1 and the LAG-3, and the affinity was not reduced.

### EXAMPLE 16 Activity of anti-LAG-3 Humanized Antibody Molecule Tested by Mixed Lymphocyte Reaction (MLR)

Fresh peripheral blood mononuclear cells (PBMCs) were separated by a density gradient centrifugation and CD14⁺ T cells were sorted by magnetic beads; and the CD14+T cells were cultured by a culture medium of 20 ng/mL of GM-CSF and 10 ng/mL of IL-4, the culture medium was changed every two days, and the cells were induced into dendritic cells (DCs) in 7-10 days. Two days before the DCs were used, 25 ng/mL of TNF-α was added to induce the DCs into mature DCs, and the mature DCs were collected and prepared into a cell suspension with a cell density of 1×10⁵ cells/mL. The CD14′ T cells were sorted from the PBMCs with magnetic beads, counted, and prepared into a cell suspension with a cell density of 1×10⁵ cells/mL. 100 μl of each of the CD14⁺ T cells and the DCs were taken and added into a 96-well plate according to a ratio of 10:1.

The 5 anti-LAG-3 humanized antibody molecules prepared in example 12, the chimeric antibody CA-1 prepared in example 9, and the anti-LAG-3 antibody provided in patent application CN105992595A were used as positive controls respectively subjected to a 4-fold gradient dilution, 8 gradients were set for each antibody, 50 μl of each antibody was taken respectively and added into a 96-well plate, after 5 days, CD¹⁴⁺ T cell proliferation was tested by CCK8, reading was performed at 450 nm/630 nm, a corresponding EC50 value was calculated, and specific data was as follows:

| | | | | | | Chimeric | DN- |
|---|---|---|---|---|---|---|---|
| Clone | HA-1 | HA-2 | HA-3 | HA-4 | HA-5 | antibody CA-1 | 105992595A |
| EC50 | 6.875 | 27.09 | 15.01 | 33.8 | 31.84 | 7.226 | 39.59 |
| (ng/ml) | | | | | | | |

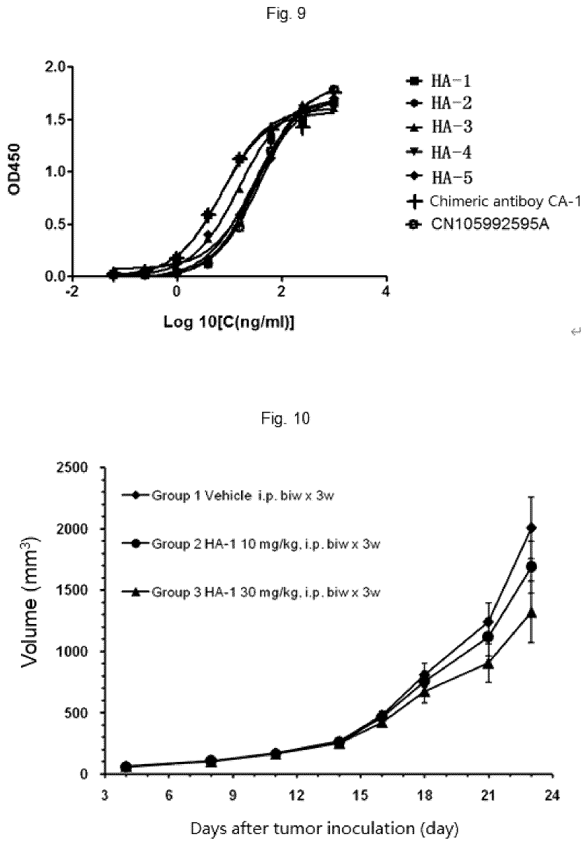

Based on the data and FIG. 9, the EC50 values of the 5 different anti-LAG-3 antibodies screened by the present disclosure were all significantly lower than that of the anti-LAG-3 antibody provided in patent application CN105992595A, which indicated that the anti-LAG-3 antibodies provided by the present disclosure all had higher activity Meanwhile the anti-LAG-3 whole antibody HA-1 had the lowest EC50 value among the 5 screened monoclonal antibodies, which indicated the antibody had a highest activity. In addition, it may be further seen from FIG. 9 that the EC50 value of the chimeric antibody CA-1 was closer to that of the anti-LAG-3 antibody HA-1, which indicated that the humanized antibody HA-1 retained the biological activity of the murine parent antibody MA-1, and the biological activity thereof was not reduced.

### EXAMPLE 17 Inhibition Test of Anti-LAG-3 Monoclonal Antibody HA-1 on MC38 Colorectal Caner in Mouse

1. Experimental animal:
species strain: Mus Musculus, NCG, mouse;
week age. 6-8 weeks; and
experimental animal provider. GemPharmatech Co.; Ltd.

2. Cell culture: MC38 tumor cells (purchased from Biovector NTCC Inc., Cat. No NTCC-MC38). The tumor cells were cultured with a DMEM containing inactivated 10% fetal bovine serum (ExCell Bio., Cat. No. FND500), 100 U/ml of penicillin, 100 μg/ml of streptomycin, and 2 mM of glutamine (purchased from ThermoFisher Scientific (China) Co., Ltd. (Giboo), Cat. No. 10566-016) in a 5% CO₂ incubator at 37° C., and passaged in vials every other 3-4 days after the cell grew full, and the tumor cells in a logarithmic growth phase were used for tumor inoculation in vivo.

3. Inoculation of tumor cells and grouping: the PBS-resuspended MC38 tumor cells with a concentration of 1.0×10⁷/mL were inoculated subcutaneously in the right rib of the experimental animal at 100p L per animal. When the tumor grew to about 61 mm³, the mice were administered in 3 groups with 8 mice in each group. The groups were a vehicle control group, HA-1 (10 mg/kg, i.p.: biw×3 w), and HA-1 (30 mg/kg, i.p., biwx3 w).

4. Detection indicators: the tumor volume was measured twice a week using a vernier caliper, the long diameter and the short diameter of the tumor were measured, and the volume calculation formula was: volume=0.5×long diameter xshort diameter², and a relationship between the change in the tumor volume and the administration time was recorded and the result was shown in FIG. 10.

Based on data in FIG. 10, the anti-LAG-3 monoclonal antibody HA-1 may inhibit tumor growth, exhibiting a dose-dependent response.

### EXAMPLE 18 Evaluation of Thermostability of Anti-LAG-3 Monoclonal Antibody HA-1 Protein Molecule

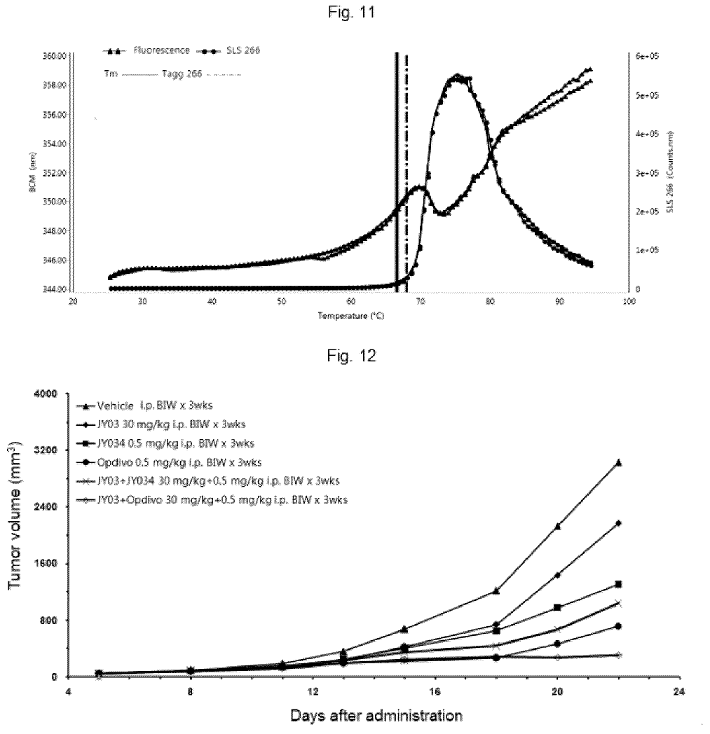

The anti-LAG-3 monoclonal antibody HA-1 protein molecule was ultrafiltered, transferred in a PBS buffer system, and centrifuged at 12,000 rpm and 4° C. for 5 min, and the thermostability of the anti-LAG-3 monoclonal antibody HA-1 protein molecule was evaluated by a multifunctional protein thermostability analysis system (purchased from Uncariained Labs). By monitoring the change of the protein endogenous fluorescence with a temperature (from 25° C., heating to 95° C. at a heating rate of 0.3° C./min), the change of a protein conformation was detected, a protein melting temperature Tm was determined and the protein conformation stability was evaluated. When a sample was aggregated, a scattered light wave was interfered and a signal of the scattered light was increased. A colloidal stability of a protein (characterized by Tagg) was determined by a static light scattering. The result was shown in the following table and FIG. 11.

| | Tempera- | Average | Tagg | Average |
|---|---|---|---|---|
| | ture | temperature | 266 | Tagg 266 |
| Sample | (° C.) | (° C.) | (° C.) | (° C.) |
| 2 mg/ml of anti-LAG-3 | 66.6 | 66.6 | 67.96 | 68.0 |
| monoclonal antibody HA-1 | | | | |
| protein molecule | 66.6 | | 68.02 | |

As shown in the above table and FIG. 11, the anti-LAG-3 monoclonal antibody HA-1 protein molecule had a temperature of 66.6° C. and an average Tagg of 68.0° C. and showed a superior conformational stability and colloidal stability.

### EXAMPLE 19

On the basis of the above example, example 19 of the present disclosure further defined the following solution: The present disclosure further provided use of the anti-LAG-3 monoclonal antibody or the antigen binding fragment thereof in combination with an anti-PD-1 monoclonal antibody in the preparation of a drug for treating a cancer or an immunological disease.

Further, the anti-PD-1 monoclonal antibody was selected from DFPD1-9, DFPD1-10, DFPD1-11, DFPD1-12, DFPD1-13, nivolumab, pembrolizumab, toripalimab, sintilimab, tislelizumab, camrelizumab, penpulimab, or zimberelimab.

The nivolumab, pembrolizumab, toripalimab, sintilimab, tislelizumab, camrelizumab, penpulimab, or zimberelimab were all marketed products.

The DFPD1-9, DFPD1-10, DFPD1-11, DFPD1-12, and DFPD1-13 were anti-PD-1 monoclonal antibodies provided by a patent with the application number of CN201510312910.8, the DFPD1-9 comprised a light chain variable region shown in SEC) ID No: 44 and a heavy chain variable region shown in SEQ ID No: 43; the DFPD1-10 comprised a light chain variable region shown in SEQ ID No: 45 and a heavy chain variable region shown in SEQ ID No: 43; the DFPD1-11 comprised a light chain variable region shown in SEQ ID Na. 44 and a heavy chain variable region shown in SEQ ID No: 46; the DFPD1-12 comprised a light chain variable region shown in SEQ ID No: 44 and a heavy chain variable region shown in SEQ ID No. 47; and the DFPD1-13 comprised a light chain variable region shown in SEQ ID No: 45 and a heavy chain variable region shown in SEQ ID Na. 46. Specific sequences were as follows:

A sequence of SEQ ID No. 43 was as follows:
QVQLVESGGGVVQPGRSLRLDCKASGITFSNYGMHWVRQAPGKGLEWVA
VIWYDSSRKYYADSVKGRFTISRDNSKNTLFLQMNSLRAEDTAVYYCAT
NNDYWGQGTLVTVSS;
A sequence of SEQ ID No. 44 was as follows:
DIQMTQSPSSLSASVGDRVTITCRASQSIHNYLDWYQQKPGKAPKLLIY
NASTRATGVPSRFSGSGSGTDFTLTISSLQPEDFATYYCQQELHLPLTF
GQGTKVEIK;
A sequence of SEQ ID No. 45 was as follows:
DIQMTQSPSSLSASVGDRVTITCRASQSVSNYLDWYQQKPGKAPKLLIY
DASTRARTGVPSRFSGSGSGTDFTLTISSLQPEDFATYYCQQNMQLPLT
FGQGTKVEIK;
A sequence of SEQ ID No. 46 was as follows:
QVQLVESGGGVVQPGRSLRLDCKASGITFSNNGMHWVRQAPGKGLEWVA
VIWYDSSRKYYADSVKGRFTISRDNSKNTLFLQMNSLRAEDTAVYYCAT
NNDYWGQGTLVTVSS;
and
A sequence of SEQ ID No. 47 was as follows:
QVQLVESGGGVVQPGRSLRLDCKASGITFSNYGMHWVRQAPGKGLEWVA
VIWYDGSKKYYADSVKGRFTISRDNSKNTLFLQMNSLRAEDTAVYYCAT
NNDYWGQGTLVTVSS.

In addition, the anti-PD-1 monoclonal antibody further comprised a heavy chain constant region and a light chain constant region. The sequences of the heavy chain constant region and the light chain constant region were the same as those of the heavy chain constant region and the light chain constant region of the anti-PD-1 monoclonal antibody provided in the patent with the application number of CN201510312910.8.

Further, the cancer was selected from leukemia, lung cancer, stomach cancer, esophageal cancer, ovarian cancer, head and neck cancer, melanoma, kidney cancer, breast cancer, colorectal cancer, liver cancer, pancreatic cancer or bladder cancer; and the immunological disease comprised psoriasis, Crohns disease, rheumatoid arthritis, primary biliary cirrhosis, systemic lupus erythematosus, multiple sclerosis, ulcerative colitis, and autoimmune hepatitis.

### EXAMPLE 20

On the basis of example 19, example 20 of the present disclosure further defined that the anti-PD-1 monoclonal antibody was DFPD1-10, and the DFPD1-10 comprised a light chain variable region shown in SEQ ID No: 45 and a heavy chain variable region shown in SEQ ID No: 43.

### EXAMPLE 21

On the basis of example 19, example 21 of the present disclosure further defined that the anti-PD-1 monoclonal antibody was nivolumab.

### EXAMPLE 22 In-Vivo Pharmaceutical Effect Experiment of Anti-LAG-3 Monoclonal Antibody in Combination with Anti-PD-1 Monoclonal Antibody for MC38 Colorectal Cancer Model

1 Test drug

(1) The anti-LAG-3 monoclonal antibody HA-1 provided in example 10 with the number of JY03;

provider: Beijing Dongfang Biotech Co., Ltd; and

quantity: 11 mL per one, 4.9 mg/mL, and a total of 7;

(2) The anti-PD-1 monoclonal antibody provided in example 20 was DFPD1-10 with the number of JY034 in the experiment;

provider: Beijing Dongfang Biotech Co., Ltd; and

quantity: 10 ml, 10 mg/ml, 3 bottles, and a total of 300 mg; and

(3) Opdivo (nivolumab)

manufacturer: Bristol Myers Squibb; and

quantity: 100 mg/10 ml, 1

2. Experimental animal

Species strain: MusMusculus, B8/JGpt-Pdcdiem/Cin (hPDCD1) Lag3 em 1 Cin (hLAG3)/Gpt

Sex: female

Week age: 6-8 weeks

Weight. 18-22 g

Quantity: 75

Experimental animal provider: GerrPharmatech Co. Ltd.

Production license number. SCXK (Su) 2018-0008

3. Experimental method

(1) Cell culture

MC38 tumor cell was cultured with a DMEM containing inactivated 10% fetal bovine serum, 100 U/mL of penicillin; 100 μg/mL of streptomycin, and 2 mM of glutamine in a 5% CO₂ incubator at 37° C., and passaged in vials every other 3-4 days after the cell grew full, and the tumor cell in a logarithmic growth phase was used for tumor inoculation in vim.

(2) Tumor cell inoculation and grouping

The PBS-resuspended MC38 tumor cells with a concentration of 1×10⁷/mL was inoculated subcutaneously in the right rib of the experimental animal at 1×10⁵/100 μL per animal. When the tumor grew to about 50 mm³, the mice were administered (the same day was marked PG-DO) in 8 groups with 10 mice in each group.

(3) Measurement of mouse weight and experiment indicators

A tumor volume was measured 2-3 times a week using a vernier caliper, a mouse weight was weighed using an electronic scale; long diameter and short diameter of the tumor were measured, and a volume calculation formula was: volume=0.5×long diameter×short diameter². The T/C value was calculated according to the tumor volume, wherein T is an average value of a relative tumor volume (RTV) of each test substance-treated group, C is an average value of a RTV of a control group, and the RTV is a ratio of the tumor volume after administration to that before administration. Tumor growth inhibition rate (TGITV) (%)=(1−T/C)×100%.

At an end of the experiment, the animals were euthanized, the tumors were stripped, weighed, placed in order, and photographed. The tumor weight inhibition rate (TGITW) (%) was calculated with a formula (1-T/C)×100%, wherein T/C=TW average value of treatment groups/TW average value of control group.

In principle, an evaluation criterion was: T/C (%)>40% was ineffective; and TC (%) was less than or equal to 40%, and P<0.05 was effective after a statistical treatment.

4. Administration regimen

| | Number | | | Admini- | |
|---|---|---|---|---|---|
| | of | | Dose | stration | Administration |
| Groups | animals | Treatment | (mg/kg)* | route | frequency |
| 1 | 10 | Vehicle | — | i.p. | biw × 3 wks |
| 2 | 10 | JY03 | 30 | i.p. | biw × 3 wks |
| 3 | 10 | JY034 | 0.5 | i.p. | biw × 3 wks^{#} |
| 4 | 10 | Opdivo | 0.5 | i.p. | biw × 3 wks^{#} |
| 5 | 10 | JY03 | 30 | i.p. | biw × 3 wks |
| | | JY034 | 0.5 | | biw × 3 wks^{#} |
| 6 | 10 | JY03 | 30 | i.p. | biw × 3 wks |
| | | Opdivo | 0.5 | | biw × 3 wks^{#} |

Note:
*an administration volume of each group was 10 μL/g according to an animal weight and the administration amount may be adjusted when the weight was reduced by 15-20%; i.p .: intraperitoneal injection; biw × 3 wks: twice weekly for three weeks, and a total of 6 times; and ^{#}a drug administration was started at a second administration time of JY03 and the actual administration time was a total of 5 times in three weeks.

5. Statistical Analysis

A statistical analysis among groups was performed on tumor volume and tumor weight using an IBM SPSS Statistics 22.0 statistical software and a one-way ANOVA test. A significant difference was considered when P<0.05.

6. Data results.

(1) Results of tumor growth inhibition:

| | | Body weight | | Tumor volume | | | Tumor | | | | | |
|---|---|---|---|---|---|---|---|---|---|---|---|---|
| | | (g) [symbol] | | (mm [symbol] ) [symbol] | | | growth | | | | | |
| | | Before | After | Belove | After | | inhibi- | | | | | |
| | Number | adminis- | adminis- | adminis- | adminis- | | tion | | | | | |
| | of | tration | tration | tration | tration | T/C | rate | | | | | |
| Groups | animals | (PG-D0) | (PG-D17) | (PG-D0) | (PG-D17) | (%) | (%) | p [symbol] | p^{c} | p^{d} | p [symbol] | p^{f} |
| Vehicle | 10 | 21.1 ± 0.4 | 25.4 ± 0.8 | 50 ± 3 | 3032 ± 577 | — | — | — | <0.001 | 0.149 | 0.001 | <0.001 |
| JY03 | 10 | 21.8 ± 0.5 | 25.2 ± 0.5 | 50 ± 3 | 2173 ± 339 | 75% | 25% | 0.149 | 0.016 | — | 0.059 | 0.002 |
| JY034 | 10 | 21.3 ± 0.3 | 24.6 ± 0.9 | 50 ± 3 | 1308 ± 487 | 42% | 58% | 0.005 | 0.316 | 0.146 | 0.653 | 0.095 |
| Opidivo | 10 | 21.2 ± 0.2 | 22.6 ± 0.7 | 50 ± 3 | 713 ± 478 | 28% | 72% | <0.001 | — | 0.016 | 0.579 | 0.497 |
| JY03 + | 10 | 21.1 ± 0.3 | 23.9 ± 0.8 | 50 ± 3 | 1041 ± 331 | 39% | 52% | 0.001 | 0.579 | 0.059 | — | 0.219 |
| JY034 | | | | | | | | | | | | |
| JY03 + | 10 | 21.0 ± 0.5 | 22.4 ± 0.5 | 49 ± 3 | 311 ± 129 | 10% | 90% | <0.001 | 0.497 | 0.002 | 0.219 | — |
| Opdivo | | | | | | | | | | | | |

Note:
A one-way ANOVA was performed on each group, a least significant difference (LSD)was used for a post hoc test;
^{a}meantstandard errors;
^{b}compared with the vehicle group;
^{c}compared with the Opdivo group;
^{d}compared with the JY03 group;
^{e}compared with the JY03 + JY034 group; and
^{f}compared with the JY03 + Opdivo group. There were no statistical differences among the JY034 treatment groups (all P > 0.05). 3, 5, 1, and 4 mice had completely regressed tumors in the JY034 group, the Opdivo group, the JY03 + JY034 group, and the JY03 + Opdivo group, respectively. [symbol]
indicates data missing or illegible when filed

(2) Tumor Weight

| | | | Tumor | | | | | |
|---|---|---|---|---|---|---|---|---|
| | Number | Tumor | weight | | | | | |
| | of | weight | Inhibition | | | | | |
| Groups | animals | (g)^{a} | rate (%) | p^{b} | p^{c} | p^{d} | p^{d} | p^{f} |
| Vehicle | 10 | 2.980 ± 0.596 | — | — | <0.001 | 0.255 | 0.003 | <0.001 |
| JY03 | 10 | 2.254 ± 0.379 | 24% | 0.255 | 0.027 | — | 0.057 | 0.002 |
| JY034 | 10 | 1.318 ± 0.500 | 56% | 0.010 | 0.443 | 0.143 | 0.655 | 0.097 |
| Opdivo | 10 | 0.830 ± 0.541 | 72% | 0.001 | — | 0.027 | 0.748 | 0.367 |
| JY03 + JY034 | 10 | 1.034 ± 0.349 | 85% | 0.003 | 0.748 | 0.057 | — | 0.223 |
| JY03 + Opdivo | 10 | 0.256 ± 0.113 | 91% | <0.001 | 0.367 | 0.002 | 0.223 | — |

Note:
A one-way ANOVA was performed on each group, a least significant difference (LSD) was used for a post hoc test;
^{a}meantstandard errors;
^{b}compared with the vehicle group;
^{c}compared with the Opdivo group;
^{d}compared with the JY03 group;
^{e}compared with the JY03 + JY034 group; and
^{f}compared with the JY03 + Opdivo group.

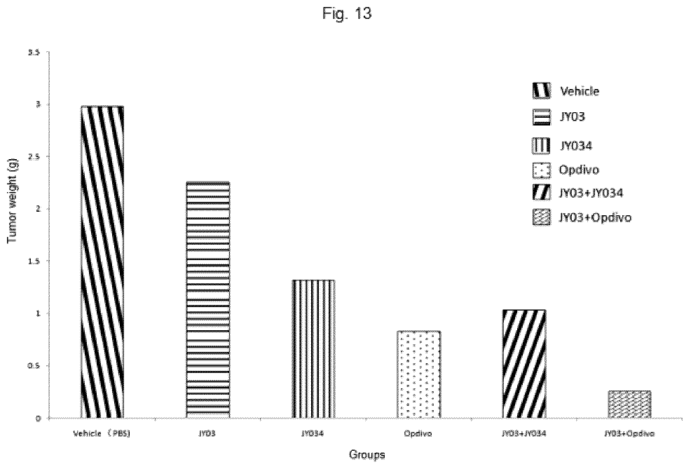

In conclusion, based on the data and FIGS. 12 and 13, in the test drug JY03 group, JY034 group, Opdivo group, JY03+JY034 group, and JY03+Opdivo group, a definite anti-tumor effect was generated on a PD-1 and LAGS double-target humanized colorectal cancer MC38 model, wherein a tumor inhibition effect of the JY034-JY034 group was obviously stronger than that of the single drug JY03 group and the single drug JY034 group, and the tumor inhibition effect of the JY03+Opdivo group was obviously stronger than that of the single drug JY03 group and the single drug Opdivo group. Therefore, the anti-LAG-3 monoclonal antibody may improve the tumor inhibition effect of the anti-PD-1 monoclonal antibody. In addition, the animals in each group had a good tolerance and no obvious adverse reaction was shown.

The present disclosure is not limited to the above optimal embodiments, and anyone may derive various other forms of products under the motivation of the present disclosure. However, regardless of any changes in their shapes or structures, any technical solution that is the same or similar to the present application fans within the scope of protection of the present disclosure.

What is claimed is:

1. An anti-LAG-3 monoclonal antibody or an antigen binding fragment thereof, comprises a heavy chain variable region and a light chain variable region, wherein the heavy chain variable region comprises 3 heavy chain complementarity determining regions, respectively represented by HCDR1, HCDR2, and HCDR3, and the light chain variable region comprises 3 light chain complementarity determining regions, respectively represented by LCDR1, LCDR2, and LCDR3; and the monoclonal antibody or the antigen binding fragment thereof is selected from any one of the following:
A-1: the heavy chain complementarily determining region HCDR1 comprises an amino acid sequence shown in SEQ ID No: 1, the heavy chain complementarity determining region HCDR2 comprises an amino acid sequence shown in SEQ ID No: 2, the heavy chain complementarity determining region HCDR3 comprises an amino acid sequence shown in SEQ ID No: 3, the tight chain complementarity determining region LCDR1 comprises an amino acid sequence shown in SEQ ID No: 4, the light chain complementarity determining region LCDR2 comprises an amino acid sequence shown in SEQ ID No: 5, and the light chain complementarily determining region LCDR3 comprises an amino acid sequence shown in SEQ ID No: 6;
A-2: the heavy chain complementarily determining region HCDR1 comprises an amino acid sequence shown in SEQ ID No: 1, the heavy chain complementarity determining region HCDR2 comprises an amino acid sequence shown in SEO ID No: 2, the heavy chain complementarity determining region HCDR3 comprises an amino acid sequence shown in SEQ ID No: 3, the light chain complementarity determining region LCDR1 comprises an amino acid sequence shown in SEQ ID No: 4, the light chain complementarity determining region LCDR2 comprises an amino acid sequence shown in SEQ ID No: 7, and the light chain complementarily determining region LCDR3 comprises an amino acid sequence shown in SEQ ID No: 8;
A-3: the heavy chain complementarity determining region HCDR1 comprises an amino acid sequence shown in SEQ ID No: 9, the heavy chain complementarity determining region HCDR2 comprises an amino acid sequence shown in SEQ ID No: 10, the heavy chain complementarity determining region HCDR3 comprises an amino acid sequence shown in SEQ ID No: 11, the light chain complementarity determining region LCDR1 comprises an amino acid sequence shown in SEQ ID No. 12, the light chain complementarity determining region LCDR2 comprises an amino acid sequence shown in SEQ ID No: 13, and the light chain complementarity determining region LCDR3 comprises an amino acid sequence shown in SEQ ID No: 14; and
A-4: the heavy chain complementarity determining region HCDR 1 comprises an amino acid sequence shown in SEQ ID No: 15, the heavy chain complementarity determining region HCDR2 comprises an amino acid sequence shown in SEQ ID No: 16, the heavy chain complementarity determining region HCDR3 comprises an amino acid sequence shown in SEQ ID No: 17, the light chain complementarity determining region LCDR1 comprises an amino acid sequence shown in SEQ ID No: 18, the light chain complementarity determining region LCDR2 comprises an amino acid sequence shown in SEQ ID No: 19, and the light chain complementarity determining region LCDR3 comprises an amino acid sequence shown in SEQ ID No: 20.

2. The anti-LAG-3 monoclonal antibody or the antigen binding fragment thereof according to claim 1, wherein the monoclonal antibody or the antigen binding fragment thereof is a murine antibody molecule, and the murine antibody molecule is selected from any one of the following:
MA-1: the heavy chain variable region comprises an amino acid sequence shown in SEQ ID No: 21, and the light chain variable region comprises an amino acid sequence shown in SEQ ID No: 22;
MA-2 the heavy chain variable region comprises an amino acid sequence shown in SEQ ID No: 21, and the light chain variable region comprises an amino acid sequence shown in SEQ ID No: 23;
MA-3: the heavy chain variable region comprises an amino acid sequence shown in SEQ ID No: 24, and the light chain variable region comprises an amino acid sequence shown in SEQ ID No: 25; and
MA-4: the heavy chain variable region comprises an amino acid sequence shown in SEQ ID No. 26, and the light chain variable region comprises an amino acid sequence shown in SEQ ID No: 27.

3. The anti-LAG-3 monoclonal antibody or the antigen binding fragment thereof according to claim 2, wherein the murine antibody molecule further comprises a heavy chain constant region and a light chain constant region, the heavy chain constant region is one of murine IgG1 type, IgG2a type, IgG2b type, and IgG3 type, the heavy chain constant region of the IgG1 type has an amino acid sequence shown in SEQ ID No 29, the heavy chain constant region of the IgG2a type has an amino acid sequence shown in SEQ ID No: 30, the heavy chain constant region of the IgG2b type has an amino acid sequence shown in SEQ ID No: 31, and the heavy chain constant region of the IgG3 type has an amino acid sequence shown in SEQ ID No 32; and the light chain constant region is a murine Ct chain with an amino acid sequence shown in SEQ ID No: 2%.

4. The anti-LAG-3 monoclonal antibody or the antigen binding fragment thereof according to claim 2, wherein the monoclonal antibody or the antigen binding fragment thereof is a chimeric antibody molecule, the chimeric antibody molecule comprises a heavy chain variable region of the murine antibody molecule, a light chain variable region of the murine antibody molecule, and a humanized antibody constant region; the humanized antibody constant region comprises a humanized antibody heavy chain constant region and a humanized antibody light chain constant region, the humanized antibody heavy chain constant region is one of human IgG1 type, IgG2 type, or IgG4 type, the humanized antibody heavy chain constant region of the IgG1 type has an amino acid sequence shown in SEQ ID No: 39, the humanized antibody heavy chain constant of the IgG2 type region has an amino acid sequence shown in SEQ ID No: 40, and the humanized antibody heavy chain constant region of the IgG4 type has an amino acid sequence shown in SEQ ID No: 41; and the humanized antibody light chain constant region is a human Ct chain having an amino acid sequence shown in SEQ ID No 42.

5. The anti-LAG-3 monoclonal antibody or the antigen binding fragment thereof according to claim 1, wherein the monoclonal antibody or the antigen binding fragment thereof is a humanized antibody molecule, and the humanized antibody molecule is selected torn any one of the following:
HA-1: the heavy chain variable region comprises an amino acid sequence shown in SEQ ID No: 33, and the light chain variable region comprises an amino acid sequence shown in SEQ ID No: 34;
HA-2: the heavy chain variable region comprises an amino acid sequence shown in SEQ ID No: 33, and the light chain variable region comprises an amino acid sequence shown in SEQ ID No: 35;
HA-3: the heavy chain variable region comprises an ammo acid sequence shown in SEQ ID No: 36, and the light chain variable region comprises an amino acid sequence shown in SEQ ID No: 35;
HA-4 the heavy chain variable region comprises an amino acid sequence shown in SEQ ID No: 36; and the light chain variable region comprises an amino acid sequence shown in SEQ ID No: 34; and
HA-5: the heavy chain variable region comprises an amino acid sequence shown in SEQ ID No: 37, and the light chain variable region comprises an amino acid sequence shown in SEQ ID No: 38.

6. The anti-LAG-3 monoclonal antibody or the antigen binding fragment thereof according to claim 5, wherein the humanized antibody molecule further comprises a heavy chain constant region and a light chain constant region, the heavy chain constant region is one of human IgG1 type, IgG2 type, or IgG4 type, the heavy chain constant region of the IgG1 type has an amino acid sequence shown in SEQ ID No: 39, the heavy chain constant region of the IgG2 type has an amino acid sequence shown in SEQ ID No: 40, the heavy chain constant region of the IgG4 type has an amino acid sequence shown in SEQ ID No 41, and the tight chain constant region is a human Cₖ chain with an amino acid sequence shown in SEC) ID No: 42.

7. The anti-LAG-3 monoclonal antibody or the antigen binding fragment thereof according to claim 5, wherein the humanized antibody molecule is a full-length antibody or an antibody fragment, the humanized antibody molecule comprises one or a combination of more of Fab, F(ab)2, Fv or ScFv.

8. A polypeptide or a protein, comprising the anti-LAG-3 monoclonal antibody or the antigen binding fragment thereof according to claim 1.

9. A polynucleotide, encoding an amino acid sequence of the anti-LAG-3 monoclonal antibody or the antigen binding fragment thereof according to claim 1.

10. A recombinant DNA expression vector; comprising the polynucleotide according to claim 9.

11. A host cell transfected with the recombinant DNA expression vector according to claim 10, wherein the host cell comprises a prokaryotic cell, a yeast cell, an insect cell, or a mammalian cell.

12. A drug, comprising the anti-LAG-3 monoclonal antibody or the antigen binding fragment thereof according to claim 1.

13. The anti-LAG-3 monoclonal antibody or the antigen binding fragment thereof according to claim 1 for use in treating a cancer or an immunological disease.

14. The anti-LAG-3 monoclonal antibody or the antigen binding fragment thereof according to claim 1 in combination with an anti-PD-1 monoclonal antibody for use in treating a cancer or an immunological disease.

15. The anti-LAG-3 monoclonal antibody or the antigen binding fragment thereof according to claim 14 in combination with the anti-PD-1 monoclonal antibody for use in treating a cancer or an immunological disease wherein the anti-PD-1 monoclonal antibody is selected from the group consisting of DFPD1-9; DFPD1-10, DFPD1-11; DFPD1-12, DFPD1-13; nivolumab, pembrolizumab, toripalimab, sintilimab, tislelizumab, camrelizumab, penpulimab, or zimberelimab.

16. The anti-LAG-3 monoclonal antibody or the antigen binding fragment thereof according to claim 15 in combination with the anti-PD-1 monoclonal antibody for use in treating a cancer or an immunological disease, wherein the anti-PD-1 monoclonal antibody is DFPD1-10, and the DFPD1-10 comprises a light chain variable region shown in SEQ ID No: 45 and a heavy chain variable region shown in SEQ ID No: 43.

17. (canceled)

18. The anti-LAG-3 monoclonal antibody or the antigen binding fragment thereof according to claim 14 in combination with the anti-PD-1 monoclonal antibody for use in treating a cancer or an immunological disease, wherein the cancer is selected from leukemia, lung cancer, stomach cancer, esophageal cancer, ovarian cancer, head and neck cancer, melanoma, kidney cancer, breast cancer, colorectal cancer, liver cancer, pancreatic cancer or bladder cancer; and the immunological disease comprises psoriasis, Crohn's disease, rheumatoid arthritis, primary biliary cirrhosis, systemic lupus erythematosus; multiple sclerosis, ulcerative colitis, and autoimmune hepatitis.

19. The anti-LAG-3 monoclonal antibody or the antigen binding fragment thereof according to claim 13 for use in treating a cancer or an immunological disease, wherein the cancer is selected from leukemia, lung cancer, stomach cancer, esophageal cancer, ovarian cancer, head and neck cancer, melanoma, kidney cancer, breast cancer, colorectal cancer, liver cancer, pancreatic cancer or bladder cancer; and the immunological disease comprises psoriasis, Crohn's disease, rheumatoid arthritis, primary biliary cirrhosis, systemic lupus erythematosus, multiple sclerosis, ulcerative colitis, and autoimmune hepatitis.

20. The host cell according to claim 11, wherein the host cell is the mammalian cell, and the mammalian cell is an HEK293E cell, a CHO cell, or an NS0 cell.


SEQUENCE LISTING (47 sequences)

SEQ ID NO: 1 - protein, 5 aa, Artificial Sequence
  Synthesized
DTYMH

SEQ ID NO: 2 - protein, 17 aa, Artificial Sequence
  Synthesized
KIDPANGNTKYDPKFQG

SEQ ID NO: 3 - protein, 8 aa, Artificial Sequence
  Synthesized
DTTVGLDY

SEQ ID NO: 4 - protein, 10 aa, Artificial Sequence
  Synthesized
RASSSVNYMY

SEQ ID NO: 5 - protein, 7 aa, Artificial Sequence
  Synthesized
YTSKLAS

SEQ ID NO: 6 - protein, 9 aa, Artificial Sequence
  Synthesized
QQWSSNPHT

SEQ ID NO: 7 - protein, 7 aa, Artificial Sequence
  Synthesized
YTSNLAP

SEQ ID NO: 8 - protein, 10 aa, Artificial Sequence
  Synthesized
QQFTSSPSMT

SEQ ID NO: 9 - protein, 7 aa, Artificial Sequence
  Synthesized
GYSFTSY

SEQ ID NO: 10 - protein, 17 aa, Artificial Sequence
  Synthesized
WIFPGTGNTRYNEKFKG

SEQ ID NO: 11 - protein, 12 aa, Artificial Sequence
  Synthesized
IGGRLTGDAMDY

SEQ ID NO: 12 - protein, 15 aa, Artificial Sequence
  Synthesized
RASESVDSYGNSFMH

SEQ ID NO: 13 - protein, 7 aa, Artificial Sequence
  Synthesized
LASNLES

SEQ ID NO: 14 - protein, 9 aa, Artificial Sequence
  Synthesized
QQNNEDPWT

SEQ ID NO: 15 - protein, 5 aa, Artificial Sequence
  Synthesized
TYAVH

SEQ ID NO: 16 - protein, 17 aa, Artificial Sequence
  Synthesized
VIWSGGSTDYNTAFISR

SEQ ID NO: 17 - protein, 8 aa, Artificial Sequence
  Synthesized
LDGTFFDY

SEQ ID NO: 18 - protein, 12 aa, Artificial Sequence
  Synthesized
TASSSVSSSYLH

SEQ ID NO: 19 - protein, 7 aa, Artificial Sequence
  Synthesized
STSNLAS

SEQ ID NO: 20 - protein, 9 aa, Artificial Sequence
  Synthesized
HQYHRSPPT

SEQ ID NO: 21 - protein, 117 aa, Artificial Sequence
  Synthesized
EVQLQQSGAELVKPGASVKLSCTASGFNIKDTYMHWVKQRPEQGLEWIGKIDPANGNTKYDPKFQGKATITADTSSNTAYLQLSRLTSEDTAVYYCARDTTVGLDYWGQGTTLTVSS

SEQ ID NO: 22 - protein, 106 aa, Artificial Sequence
  Synthesized
DIVITQSTAIMSASLGEKVTMSCRASSSVNYMYWYQQKSDASPKLWIYYTSKLASGVPARFSGSGSGTSYSLTISSMEAEDAATYYCQQWSSNPHTFGGGTKLEIK

SEQ ID NO: 23 - protein, 107 aa, Artificial Sequence
  Synthesized
DIVLTQTPAIMSASLGEKVTMSCRASSSVNYMYWYQQKSDASPKLWIYYTSNLAPGVPARFSGSGSGNSYSLTISSMEGEDAATYYCQQFTSSPSMTFGGGTKLEIK

SEQ ID NO: 24 - protein, 121 aa, Artificial Sequence
  Synthesized
EVKLEESGPELVKPGASVKISCKASGYSFTSYYIHWVKQRPGQGLEWIGWIFPGTGNTRYNEKFKGKATLTADTSSSTVYMQLSGLTSEDSAVYFCARIGGRLTGDAMDYWGQGTSVTVSS

SEQ ID NO: 25 - protein, 111 aa, Artificial Sequence
  Synthesized
DIVLTQSTASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIK

SEQ ID NO: 26 - protein, 116 aa, Artificial Sequence
  Synthesized
QVKLEESGPGLVQPSQSLSITCTVSAFSLTTYAVHWVRQSPGKGLEWLGVIWSGGSTDYNTAFISRLNITKDNSKSQVFFKMNSLQANDTAIYYCARLDGTFFDYWGQGTTLTVSS

SEQ ID NO: 27 - protein, 108 aa, Artificial Sequence
  Synthesized
DIVLTQTPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLEIK

SEQ ID NO: 28 - protein, 106 aa, Artificial Sequence
  Synthesized
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - protein, 323 aa, Artificial Sequence
  Synthesized
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 30 - protein, 330 aa, Artificial Sequence
  Synthesized
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 336 aa, Artificial Sequence
  Synthesized
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 32 - protein, 399 aa, Artificial Sequence
  Synthesized
ATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPELELNETCAEAQDGELDGLWTTITIFISLFLLSVCYSASVTLFKVKWIFSSVVQVKQTAIPDYRNMIGQGA

SEQ ID NO: 33 - protein, 117 aa, Artificial Sequence
  Synthesized
QVQLVQSGAEVKKPGASVKVSCKASGYTFTDTYMHWVRQAPGQGLEWIGKIDPANGNTKYDPKFQGRATITADTSISTAYMELSRLRSDDTAVYYCARDTTVGLDYWGQGTLVTVSS

SEQ ID NO: 34 - protein, 106 aa, Artificial Sequence
  Synthesized
DIVITQSPAFLSVTPGEKVTITCRASSSVNYMYWYQQKPDASPKLWIYYTSKLASGVPSRFSGSGSGTDYTFTISSLEAEDAATYYCQQWSSNPHTFGGGTKVEIK

SEQ ID NO: 35 - protein, 106 aa, Artificial Sequence
  Synthesized
DIVITQSPAFLSVTPGEKVTITCRASSSVNYMYWYQQKPDQAPKLLIYYTSKLASGVPSRFSGSGSGTDYTFTISSLEAEDAATYYCQQWSSNPHTFGGGTKVEIK

SEQ ID NO: 36 - protein, 117 aa, Artificial Sequence
  Synthesized
QVQLVQSGAEVKKPGASVKVSCKASGYNITDTYMHWVRQAPGQGLEWIGKIDPANGNTKYDPKFQGRATITADTSISTAYMELSRLRSDDTAVYYCARDTTVGLDYWGQGTLVTVSS

SEQ ID NO: 37 - protein, 117 aa, Artificial Sequence
  Synthesized
QVQLVQSGAEVKKPGASVKVSCKASGYNIKDTYMHWVKQAPGQGLEWIGKIDPANGNTKYDPKFQGKATITADTSISTAYMELSRLRSDDTAVYYCARDTTVGLDYWGQGTLVTVSS

SEQ ID NO: 38 - protein, 106 aa, Artificial Sequence
  Synthesized
DIVITQSPAFLSVTPGEKVTITCRASSSVNYMYWYQQKPDASPKLWIYYTSKLASGVPARFSGSGSGTSYTFTISSLEAEDAATYYCQQWSSNPHTFGGGTKVEIK

SEQ ID NO: 39 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 326 aa, Artificial Sequence
  Synthesized
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - protein, 327 aa, Artificial Sequence
  Synthesized
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 107 aa, Artificial Sequence [sequence omitted]

SEQ ID NO: 43 - protein, 113 aa, Artificial Sequence
  Synthesized
QVQLVESGGGVVQPGRSLRLDCKASGITFSNYGMHWVRQAPGKGLEWVAVIWYDSSRKYYADSVKGRFTISRDNSKNTLFLQMNSLRAEDTAVYYCATNNDYWGQGTLVTVSS

SEQ ID NO: 44 - protein, 107 aa, Artificial Sequence
  Synthesized
DIQMTQSPSSLSASVGDRVTITCRASQSIHNYLDWYQQKPGKAPKLLIYNASTRATGVPSRFSGSGSGTDFTLTISSLQPEDFATYYCQQELHLPLTFGQGTKVEIK

SEQ ID NO: 45 - protein, 107 aa, Artificial Sequence
  Synthesized
DIQMTQSPSSLSASVGDRVTITCRASQSVSNYLDWYQQKPGKAPKLLIYDASTRATGVPSRFSGSGSGTDFTLTISSLQPEDFATYYCQQNMQLPLTFGQGTKVEIK

SEQ ID NO: 46 - protein, 113 aa, Artificial Sequence
  Synthesized
QVQLVESGGGVVQPGRSLRLDCKASGITFSNNGMHWVRQAPGKGLEWVAVIWYDSSRKYYADSVKGRFTISRDNSKNTLFLQMNSLRAEDTAVYYCATNNDYWGQGTLVTVSS

SEQ ID NO: 47 - protein, 113 aa, Artificial Sequence
  Synthesized
QVQLVESGGGVVQPGRSLRLDCKASGITFSNYGMHWVRQAPGKGLEWVAVIWYDGSKKYYADSVKGRFTISRDNSKNTLFLQMNSLRAEDTAVYYCATNNDYWGQGTLVTVSS